(19) United States
(12) Patent Application Publication
(10) Pub. No.: US 2009/0062913 A1
(43) Pub. Date: Mar. 5, 2009

(54) Light powered microactuator, microfluidic dispenser and retinal prosthesis

(76) Inventors: Laxminarayana Saggere, Chicago, IL (US); Mandar Deshpande, North Aurora, IL (US); David Schneeweis, Downers Grove, IL (US)

Correspondence Address: GREER, BURNS & CRAIN, 300 S WACKER DR, 25TH FLOOR, CHICAGO, IL, 60606, US

(21) Appl. No.: 11/998,220

(22) Filed: Nov. 29, 2007

Related U.S. Application Data
(63) Continuation-in-part of application No. 11/663,401, filed on Aug. 30, 2007.

(51) Int. Cl. A61F 2/14

(52) U.S. Cl. 623/6.63

(57) ABSTRACT

A light powered microactuator device of the invention is an integrated device including a solar cell that provides sufficient energy to actuate an electroactive thin film coupled to a thin membrane. The lateral strain response of the electroactive thin film causes the thin membrane to move, providing an actuation force that can be applied in a wide variety of microactuator devices. A preferred embodiment light powered microactuator device of the invention includes a substrate that defines a flexible membrane in a portion thereof. An electroactive thin film is coupled to the flexible membrane such that lateral strain in the electroactive membrane causes flexing of the membrane. An integrated solar cell converts light to voltage that is applied to the electroactive thin film by an integrated electrode. A preferred embodiment device is a retinal prosthesis including one or and array of light powered microactuator devices of the invention configured to dispense a neurotransmitter chemical fluid in response to light received at the human retina.

### RELATED APPLICATION AND PRIORITY CLAIM

This application is a continuation-in-part of and claims priority under 35 U.S.C. §120 from prior application Ser. No. 11/663,401, filed Mar. 21, 2007, which application claimed priority pursuant to applicable statutes and treaties, including 35 U.S.C §119, PCT Article 8, and the Paris Convention based upon prior PCT Application Serial Number PCT/US2005/033902, filed Sep. 22, 2005, U.S. Provisional application 60/612,099, filed Sep. 22, 2004, and U.S. Provisional Application 60/613,815, filed Sep. 28, 2004.

### FIELD

A field of the invention is microactuator devices. Another field of the invention is microfluidics. A wide range of applications can use a microactuator device of the invention. Exemplary applications for a microactuator device of the invention include, but are not limited to, individual and arrays of light powered microactuators acting as switches, valves, grippers, shutters, pumps, and dispensers, and movers. A preferred exemplary embodiment is a retinal prosthesis.

### BACKGROUND

### Microactuators

Microactuators have been developed that rely upon electrical energy, such as supplied by a voltage source. However, in some applications it is inconvenient to provide a voltage source. Light driven microactuators have been studied as an alternative, with the goal of converting light energy to an electrical voltage that can activate the response of the microactuator. In a typical application, a microactuator device is connected to a discrete solar cell array. In many applications, the available power of light to stimulate response and the relatively low efficiency of solar cells that convert the light to voltage require that multiple solar cells be used to stimulate the microactuator response. In many instances, the low level of voltage output from a single solar cell requires the combination of outputs from multiple cells to drive typical microactuator devices. This then requires that multiple solar cells be used in series to power a microactuator device, increasing the footprint and/or volume occupied by the microactuator with its separate solar cells.

A variety of light driven microactuators have been studied and proposed for several applications, including, for example autonomous microsystems such as microrobots, independent space explorers, actuation in hazardous environments such as explosive or high electromagnetic interference (EMI) environments, implantable medical sensors and optical fiber communication. However, the use of light energy to actuate and dispense fluids in a controlled manner at micro scale has received little attention in the literature, perhaps because fluids present a high level of resistance to actuator movement. Many applications would benefit from a reduced threshold light powered microactuator.

### Retinal Prosthesis

Eye diseases that damage the photo-receptors cause irreversible loss of vision. Retinitis Pigmentosa (RP) refers to inherited eye diseases that cause the degeneration of photoreceptor cells in the retina. Photoreceptor cells capture and process light. As these cells degenerate and die, patients experience progressive vision loss and total loss of vision. Macular degeneration and other diseases produce similar photoreceptor degeneration and the associated loss of vision.

Age-related macular degeneration (ARMD) and Retinitis Pigmentosia are the leading causes of blindness in the world. Vision loss due to these diseases is ultimately due to degeneration of the retinal photoreceptor cells, the cells that normally absorb light entering the eye and produce a neurotransmitter chemical that eventually conveys electrical signals to the brain's primary visual cortex where vision is interpreted. Currently, there are no cures or effective treatment for photoreceptor degeneration, but a number of research efforts are currently underway worldwide to develop a retinal prosthesis that would restore at least partial vision in the people blinded by these diseases.

Current research efforts regarding a retinal prosthesis are principally directed toward electrical stimulation of the neurons. Research is promising, but the electrical approach has inherent limitations as the amount and type of vision that can be restored. While electrical stimulation is conceptually simple, it cannot discriminate various types of cells found in the retina, it requires auxiliary power supply, and the current electrode technology does not have the high spatial resolution required for a good vision. Use of batteries is not desirable as they can supply only a finite amount power and are not suited for implantation. Some retinal prostheses use microphotodiodes that convert light energy from images to electrical impulses. However, studies have concluded that the amount of visible light that can reach the subretinal device is very limited and insufficient to generate enough current to activate the microphotodiodes. Another problem with electrical stimulation is that it creates an electrochemical reaction that dissolves the materials of interconnects and wires. There are also other issues associated with the safety of electrical stimulations and stimulation threshold (e.g. epiretinal stimulation requires charge levels higher than the established safe limits for neural tissue and heat generation from the stimulus charge causes damage to retina).

### SUMMARY

A light powered microactuator device of the invention is an integrated device including a solar cell that provides sufficient electrical energy to actuate an electroactive thin film coupled to a flexible structure defined by a portion of the substrate. The lateral strain response of the electroactive thin film causes the flexible structure to move, providing an actuation force and displacement that can be applied in a wide variety of microactuator applications. A preferred embodiment of the light-powered microactuator device of the invention includes a substrate that defines a flexible membrane in a portion thereof. An electroactive thin film is coupled to the flexible membrane such that lateral strain in the electroactive thin film causes flexing of the flexible membrane. An integrated solar cell converts light to voltage that is applied to the electroactive thin film by an integrated electrode. Electroactive materials, as used herein, are any materials that produce mechanical strain in response to electrical potential, and can be formed into thin films, e.g. piezoelectric materials and electroactive polymers.

A preferred embodiment device is a retinal prosthesis including one or an array of light-powered microactuator devices of the invention configured as one or an array of microfluidic dispensers to dispense a stored neurotransmitter in response to light received at the human retina.

Another preferred embodiment of the device is a drug delivery device either implanted in the body or placed outside the body, comprising one or an array of microfluidic dispensers and one or an array of light powered microactuator devices of the invention or part thereof, configured to dispense a stored pharmacological agent in response to an externally regulated light intensity.

### BRIEF DESCRIPTION OF THE DRAWINGS

FIGS. 1A-1C illustrate a preferred embodiment light powered microactuator device of the invention;

FIGS. 2A-2F illustrate steps used for formation of an experimental solar cell having the configuration of FIGS. 1A-1C;

FIGS. 3A-3F illustrate steps used for formation of an experimental thin film electroactive actuator coupled to a flexible membrane integrated in a silicon wafer having the configuration of FIGS. 1A-1C;

FIG. 4 shows the measured V_{oc} generated by an experimental solar cell plotted against illumination;

FIG. 5 shows measured center deflection versus voltage of an experimental thin film electroactive actuator coupled to a flexible membrane integrated in a silicon wafer having the configuration of FIGS. 1A-1C;

FIGS. 6A and 6B are schematic cross section view of a preferred embodiment retinal prosthesis device using a light powered microactuator device of the invention as a microfluidic pump; FIG. 6A shows a non-actuated state and FIG. 6B shows an actuated state;

FIG. 7 shows the characteristic J-V curve for a solar cell with η_{cell}=16% and J_{sc}⁰=33.94 mA/cm²;

FIG. 8 shows a cross-sectional schematic of a circular electroactive microactuator used to discuss design criteria for a retinal prosthesis device and light powered microfluidic pump of the invention;

FIG. 9 schematically shows a convention used to describe a microactuator for design optimization treated as a composite plate with layers of unequal radii, namely the microactuator is treated as a superposition of two plates: an outer annular plate and an inner composite plate;

FIGS. 10A and 10B are top and side views of an example prototype integrated light powered microactuator of the invention, along with example dimensions and materials for the prototype;

FIG. 11 shows an experimental set up for testing of prototypes consistent with FIGS. 10A and 10B;

FIG. 12 shows center point deflection versus voltage for three prototypes consistent with FIGS. 10A and 10B;

FIG. 13 shows photovoltage versus irradiance for the three prototypes;

FIG. 14 shows displacement versus irradiance for the three prototypes;

FIG. 15 shows a model circuit of a non ideal diode for a solar cell;

FIG. 16 shows measured I-V curves for prototype 2 at irradiances of 1-10 mW/m²;

FIGS. 17A and 17B respectively show short circuit current and open circuit voltage versus irradiance for experimental and model data;

FIG. 18 shows an analytical model used to validate experimental data;

FIGS. 19A and 19B respectively show voltage build-up across the microactuator and current flowing to charge the PZT thin film;

FIGS. 20A and 20B show experimental-model correlations for input-output characteristics of an experimental microactuator as functions of photovoltage and irradiance, respectively;

FIG. 21 shows normalized photovoltage values at various irradiances;

FIG. 22 shows microactuator response time as a function of input irradiance;

FIG. 23 shows calculated net volume displacement as a function of input irradiance;

FIG. 24 shows calculated block pressure as a function of irradiance; and

FIG. 25 shows typical calculated microactuator performance curves.

### DETAILED DESCRIPTION OF THE PREFERRED EMBODIMENTS

A light powered microactuator device of the invention is an integrated device including a solar cell that provides sufficient energy to actuate an electroactive thin film coupled to a thin flexible structure, e.g. a membrane or cantilever, defined by a portion of a substrate. The lateral strain response of the electroactive thin film causes the thin flexible structure to move, providing an actuation force that can form the basis for a wide variety of microactuator devices. A preferred embodiment light powered microactuator device of the invention includes a substrate that defines a flexible membrane in a portion thereof. Silicon is a preferred substrate material, but other exemplary materials include semiconductors and soft polymers that can be micro-machined to achieve micron-scale thickness and sub-millimeter diameters of the flexible membrane. An electroactive thin film is coupled to the flexible membrane such that lateral strain in the electroactive thin film causes flexing of the flexible membrane. An integrated solar cell converts light to voltage that is applied to the electroactive thin film by an integrated electrode.

A light powered microactuator array of the invention includes a plurality of light powered microactuator devices. Each integrated device in the array includes a solar cell that provides sufficient energy to actuate an electroactive thin film coupled to a flexible membrane. The solar cell converts light to voltage that is applied to the electroactive thin film by an integrated electrode.

The integrated solar cell in light powered microactuator devices and arrays of the invention provides a self-contained power source that can be modulated by incident light or generated light. Flexible structure movement can be applied to induce mechanical movement and achieve a wide range of effects include switching, valving, gripping, shuttering, pumping, dispensing, and moving. Additionally, flexible structure movement can provide sensing, as the movement of the flexible structure in response to light can be detected. In preferred embodiments, the integrated structure with the electroactive thin film and flexible membrane responds to a small power density of light on the surface of the solar cell. This permits use of light powered microactuator devices and arrays of the invention in environments where a strong modulation signal is unavailable.

For example, the scale and response of light powered microactuator devices and arrays of the invention permits response to the available light in the human retina. The light energy available at the retina is in the range of 0.1-3 W/m². Solar cells have been used to power MEMs devices, but conventional solar powered MEMs devices typically use an array of several serially connected solar cells to produce high voltages required for their application to much higher input light levels than is available to a retinal prosthesis because the voltage generated by a single solar cell is typically insufficient for driving microactuators. For instance, the voltage output generated by single-crystal silicon solar cell is limited by the electronic bandgap of silicon, and cannot exceed 700 mV. However, the expected theoretical voltage output from a typical solar cell for the low illumination intensity in the range of 0.1-3 W/m² does not exceed 450 mV.

A preferred embodiment devices is a retinal prosthesis including one or an array of light powered microactuator devices of the invention configured to dispense a neurotransmitter chemical fluid in response to light received at the human retina. The flexible membrane acts as a diaphragm that exerts force upon the neurotransmitter chemical fluid. The substrate defines a chamber volume that encloses the neurotransmitter chemical fluid. One or more micronozzles serve as outlet for the dispensed fluid. When irradiated by light, the flexible membrane/diaphragm actuator provides pressure to dispense neurotransmitter chemical fluid through the micronozzles. The amount of fluid ejected and the rate of ejection are regulated by the intensity and the irradiation time/frequency of the incident light. A retinal prosthesis device of the invention replaces the natural mechanism of intercellular communication in neurons, namely the chemical synapse. It is the chemical stimulation from damaged presynaptic neurons that is actually lost due to diseases that cause degeneration of the photoreceptors. A retinal prosthesis in accordance with the invention dispenses neurotransmitters to focally and selectively stimulate neurons, and uses light naturally entering the eye to power the prosthetic device.

Another preferred embodiment device is a drug delivery device either implanted in the body or placed outside the body, comprising one or an array of light powered microfluidic dispensers of the invention, configured to dispense a stored pharmacological agent in response to an externally regulated light intensity.

Exemplary embodiment light powered microactuator devices, and exemplary embodiment retinal prosthesis devices will now be discussed with reference to the drawings. Drawing figures may be presented schematically, and are not to scale, but will be understood by artisans. Features may be exaggerated for purposes of illustration. Additional aspects of the invention will be apparent to artisans from the features of the specific embodiments.

### Light Powered Microactuator Device

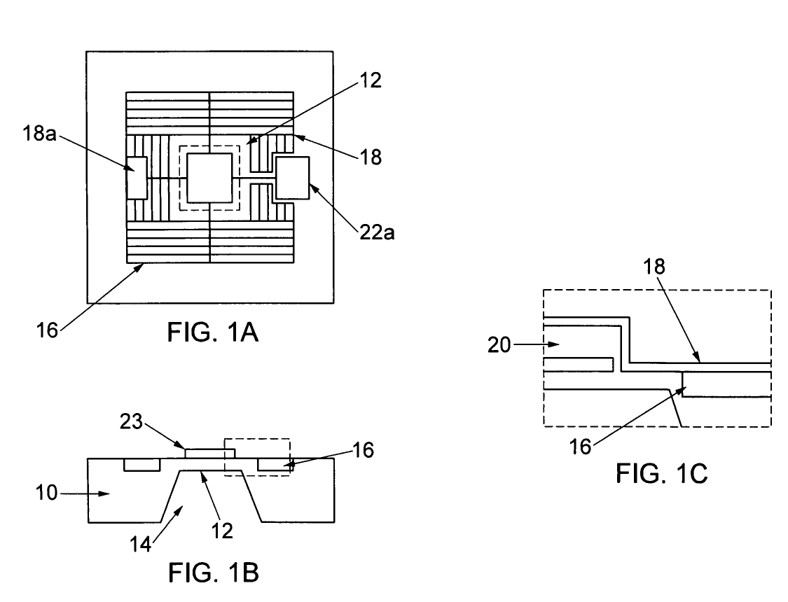

FIGS. 1A-1C illustrate a preferred embodiment light powered microactuator device of the invention. A portion of a substrate 10, e.g., a p-type silicon substrate, defines a flexible membrane 12 formed by a material removal process. A volume 14 is defined by the material removed to form the flexible membrane 12. The shape of the flexible membrane 12 and the corresponding volume 14 are generally arbitrary, and limited only by the capabilities of the material removal process employed to form the flexible membrane 12. Wet etching processes, for example, can be used to create square flexible membranes. More sophisticated processes, such as deep reactive ion etching (DRIE), can be used to form circular flexible membranes. A circular flexible membrane results in a generally cylindrical volume. In certain applications, including preferred embodiment microfluidic pump devices, a circular flexible membrane and corresponding cylindrical volume are advantageous. Circular flexible membranes with cylindrical volumes are likely to have lesser stress concentrations and lesser built-in stresses due to fabrication processes than square flexible membranes with corresponding volumes.

An n-type doped region 16 forms a p-n junction that acts as a solar cell integrated in the substrate 10. Charge collected by the solar cell 16 is communicated through a top electrode 18 that establishes a voltage potential across an electroactive thin film 20 with respect to a bottom electrode 22. In FIG. 1A, the bottom electrode 22 and top electrode 18 include test contacts 22a and 22b, used in an experiments, but unnecessary to device operation. The electroactive film 20 can be a thin active layer of PZT (Lead Zirconate Titanate (PbZrₓTi₁₋ₓO₃ where 0<x<1)) and can be applied, for example, by spin coating upon the flexible membrane 12 after formation of the bottom electrode 20. The output of the solar cell 16 is collected by the top electrode 18, part of which is formed as a grid over the solar cell 16. The top electrode 18, electroactive thin film 20 and bottom electrode 22 together form an electroactive actuator. The bottom electrode 22 is at a common potential with the p-type silicon substrate 10. Lateral strain in the electroactive thin film 20 induced by a voltage potential causes actuation of the flexible membrane 12. Flexible membrane movement can be used to accomplish a wide range of functions, as will be appreciated by artisans. A plurality of light powered microactuator devices in accordance with FIGS. 1A-1C can be formed in a single silicon substrate to create an array of light powered microactuator devices.

Experimental Data

Laboratory experiments and simulations have been conducted to test light powered microactuator devices of the invention. The experiments and simulations will be discussed. Fabrication processes used in the experiments can be modified for commercial-scale manufacturing, as will be appreciated by artisans.

A goal of the experiments was to verify operational characteristics of the light powered microactuator devices. For testing purposes and to characterize the solar cell and the electroactive microactuator individually for component study, solar cells and PZT thin film actuators were fabricated separately using the same set of masks corresponding to the integrated design shown in FIGS. 1A-1C. Experimental fabricated solar cells had same geometry and configuration as on the integrated design, but lacked the electroactive microactuator. Experimental fabricated electroactive microactuator had same geometry and configuration, but lacked the solar cell. Separate fabrication permitted detailed studies of the stand-alone performances of the solar cell and electroactive microactuator to verify response to operation conditions of interest. Additional experiments have demonstrated successful fabrication of the complete integrated light powered microactuator device including the electroactive microactuator and the solar cell.

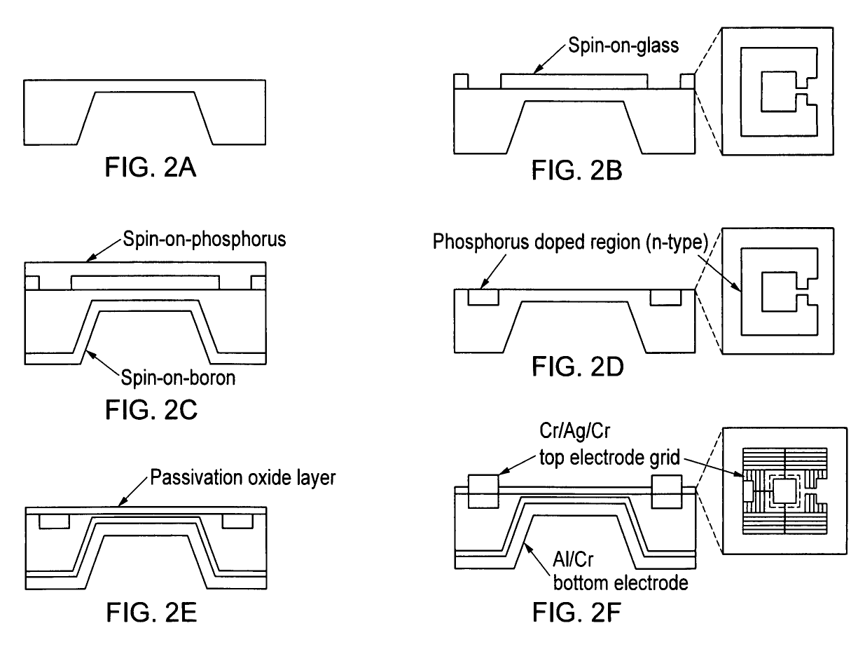

FIGS. 2A-2F illustrate steps used for formation of an experimental solar cell having the configuration of FIGS. 1A-1C. FIG. 2A shows a silicon wafer (10 Ω-cm (100) p-type silicon wafer) that has been etched to define a flexible membrane and associated volume. In FIG. 2B, a diffusion mask is created, with the pattern shown in the inset to the right of FIG. 2B. The mask is created with a spin-on-glass diffusion mask. The spin-on-glass (SOG 20B, Filmtronics Inc., Butler, Pa.) is deposited by spinning at 5000 rpm, followed by a 20-minute bake at 200° C. and 2 minutes at 350° C. on a hotplate to evaporate the solvents. The glass layer is etched in BOE with photoresist masking. In FIG. 2C, a phosphorus dopant source (n-dopant) is spin-coated (Spin-on-phosphorus P-509, Filmtronics Inc., Butler, Pa.) onto the patterned glass layer at 5000 rpm, followed by 20 minutes bake at 200° C. and 2 minutes at 350° C. on a hotplate. Additionally, a boron dopant source (Spin-on-boron B-155, Filmtronics Inc., Butler, Pa.) is spin-coated at 5000 rpm on the backside to form a back surface field (BSF) followed by a similar baking cycle. The doping diffusion is performed in a horizontal tube diffusion furnace at 900° C. for 15 minutes in an N₂—O₂ atmosphere. The residual glass products are etched off in 49% HF, resulting in the n-type doped solar cell regions seen in FIG. 2D, with the pattern shown in the inset to the right of FIG. 2D.

A thin dry oxide passivation layer is grown in a furnace at 1000° C. for 15 minutes in O₂—N₂ atmosphere, as seen in FIG. 2E. Photoresist is pattered on the device side for lift-off patterning of a top metal electrode grid. Before deposition of the metal, the oxide layer in the area beneath the metal grid area is etched in 10:1 BOE. A Cr/Ag/Cr (10 nm/100 nm/10 nm) layer is then deposited on the front side, and Cr/Al is deposited on the backside of the wafer. The top grid electrode is then patterned by standard lift-off procedure to form the pattern shown in FIG. 2F. Finally, individual solar cell chips are diced.

Example perimeter size of experimental integrated solar cells were 5 mm×5 mm. The individual solar cells with integrated flexible membranes can be made much smaller with the same or similar fabrication processes, as will be recognized by artisans. In an exemplary experimental solar cell with integrated flexible membrane and electrode, the n-doped region is confined to the extent of the metal grid. The net area on the experimental solar cell where the photon absorption can occur is calculated to be 12 mm².

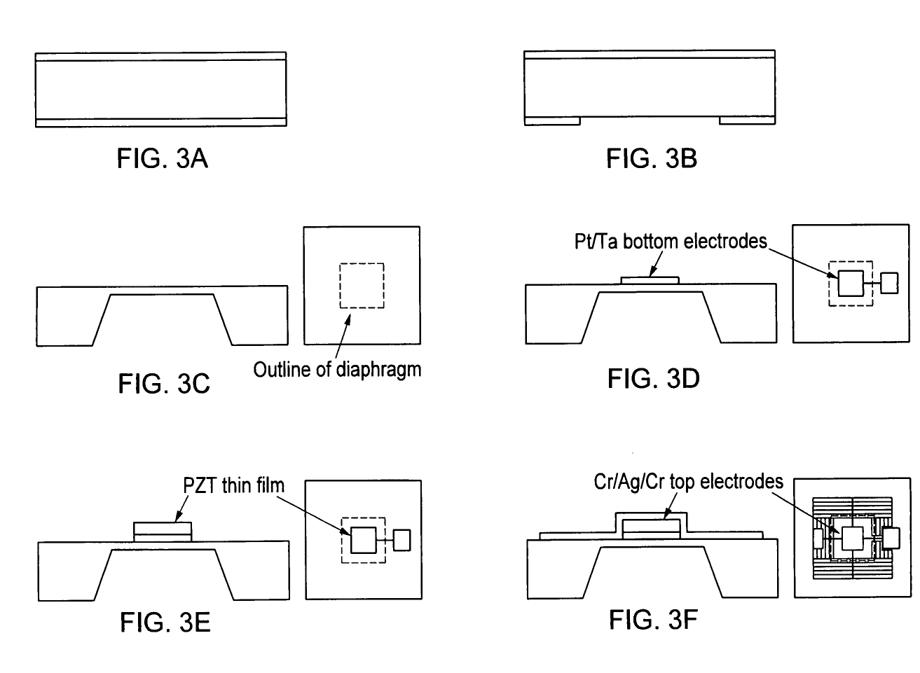

FIGS. 3A-3F illustrate steps used for formation of an experimental thin film electroactive actuator coupled to a flexible membrane integrated in a silicon wafer having the configuration of FIGS. 1A-1C. With reference to FIG. 3A-3C, silicon membranes are anisotropically etched using KOH with patterned oxide and nitride layer masking. To start with, wet-oxide 0(500 nm) is grown by steam oxidation in a furnace at 1100° C. This is followed by LPCVD deposition of 100 nm silicon nitride (FIG. 3A). The nitride is patterned using RIE (CF₄+O₂ plasma), using a positive photoresist mask, followed by patterning of the oxide in 6:1 BOE (FIG. 3B). Wet anisotropic etching is carried out in 45% KOH solution at a temperature of 85° C. An etch rate of 0.82 μm/min was measured. The etch depth was intermittently monitored under an optical profilometer (Veeco Instruments, Woodbury N.Y.) to obtain membrane thickness of 10-15 μm (FIG. 3C). After formation of the membrane, the electroactive thin film actuator is coupled to the membrane, as illustrated in FIGS. 3D-3F. After stripping the nitride and etching the oxide layer down to 100 nm, Pt/Ta (100 nm/10 nm) bottom electrode layers are deposited using e-beam evaporation and patterned by lift-off technique (FIG. 3D). Tantalum was chosen as the adhesion layer over the generally used titanium because titanium showed delamination problems of the electrode layers after deposition of PZT sol-gel exceeding approximately 350 nm in thickness.

Then, a thin-film of PZT sol-gel (52/48 10% PZT-E solution obtained from Mitsubishi Materials Corp.) was deposited using sol-gel method. The sol-gel is spread at 600 rpm for 3 seconds and spun at 3500 rpm for 30 seconds. Each layer is pyrolized at 350° C. on a hotplate for 5 mins. The film is patterned by wet-etching after every three layers (before crystallization) in 10:1 BOE followed by a 30 second dip in 2:1 HCl. The patterned layers are crystallized using RTA at 650° C. for 90 seconds. The process is repeated to obtain a 600 nm thick PZT thin film (FIG. 3E). Finally, the Cr/Ag/Cr top electrodes are deposited using e-beam evaporation and patterned using lift-off technique (FIG. 3F).

Testing

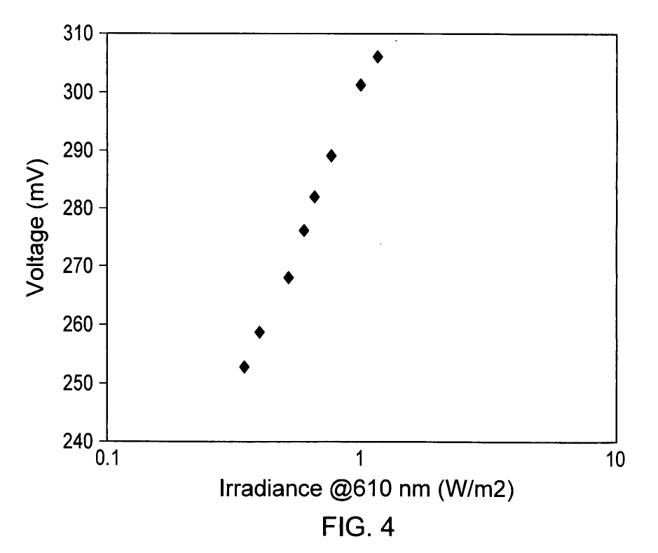

To demonstrate the feasibility of driving the microactuator using the voltage generated by the solar cell under low illumination levels of 0.1-3 W/m² testing was conducted. The open circuit (OC) voltage, V_{oc}, generated by the solar cell was tested under light irradiances ranging from 0.6-1.16 W/m². The voltage was measured using a Keithley 6514 electrometer, while the light from a standard fluorescent tube is irradiated on the sample. The irradiance, measured using a Newport 835 optical power meter, was varied using a combination of neutral density optical attenuation filters (Melles Griot Optics). FIG. 4 shows the measured V_{oc} generated by the solar cell plotted against the illumination. As expected, the V_{oc} varies logarithmically with irradiance.

To confirm the electroactive crystallization in a fabricated sample, the crystallization data for the PZT thin film was obtained by θ-2θ X-ray diffraction measured using a Siemens D-5000 powder diffractometer. The diffraction data showed that an example PZT thin film had a preferred (110) orientation.

The samples were then poled at 125° C. for 10 minutes under a field of 200 kV/cm. To confirm the electroactive behavior in the microactuator, the hysteresis loop is measured using a Sawyer-Tower circuit, with a capacitance of 200 nF at a 1 kHz. Measured loops showed a high saturation field of 360 kV/cm and a remnant polarization, Pᵣ=5 μC/cm², confirming successful fabrication.

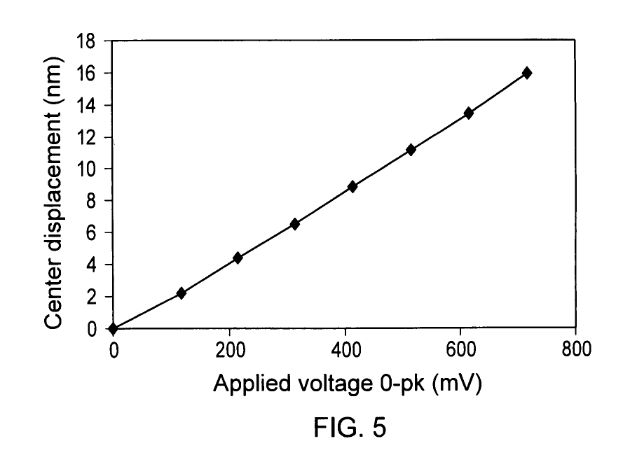

Experimental thin film PZT microactuators were excited using a biased (0-peak) sinusoidal actuation voltage signal (100 Hz) of peak magnitude ranging from 100-700 mV. The center deflection of the microactuator is measured using a microscope scanning vibrometer, (model MSV-300 from Polytec Instruments). The actuation frequency is chosen to be much smaller than the resonance frequency of the actuator (˜70 kHz). FIG. 5 shows measured center deflection of an experimental integrated thin film PZT-silicon flexible membrane actuator. As shown in FIG. 5, a center deflection of the actuator in the range of 2-16 nm was measured for applied voltages in the range of 100-700 mV.

Additional testing used an experimental setup to test actuation of an experimental integrated thin film PZT-silicon flexible membrane actuator with an experimental solar cell in series with a signal generator. The open circuit voltage generated by the solar cell is measured with a Keithley 6514 Electrometer. A microscope scanning vibrometer, which has a very high resolution for displacement measurements was used for displacement measurements, and since it only measures dynamic displacement, a modulated voltage is applied across the actuator to facilitate the use of MSV in this experiment. Results confirm that the center displacement of the actuator varies as a linear function of the open circuit voltage generated by the solar cell. Additionally, the variation of the center deflection with the incident light irradiance follows the same trend as the open circuit voltage generated by the solar cell.

### Self-Contained Retinal Prosthesis

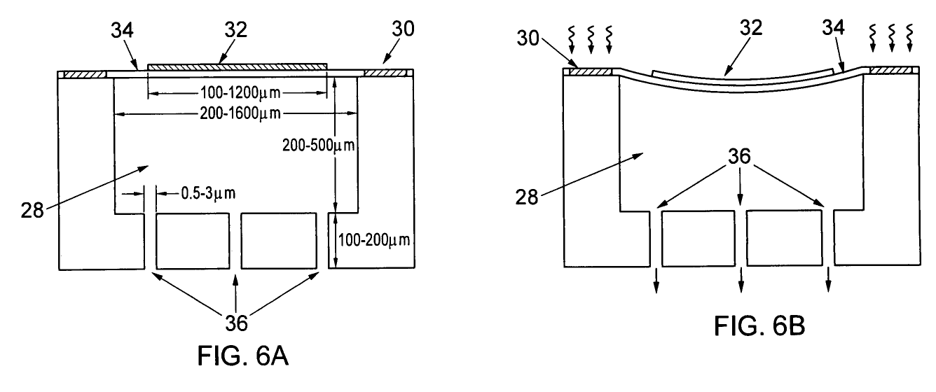

A preferred embodiment retinal prosthesis device using a light powered microactuator device of the invention as a microfluidic pump is shown in FIGS. 6A and 6B. The dimensions shown in FIGS. 6A and 6B are exemplary and are included primarily for the additional purpose of considering design optimization principles to be discussed below.

With reference to FIGS. 6A and 6B, the retinal prosthesis device includes is an integrated structure generally consistent with the light powered microactuator of FIGS. 1A-1C. In FIGS. 6A and 6B, an internal volume 28 contains a neurotransmitter chemical fluid that is ejected in response to light received at the human retina. Light incident on an integrated solar cell 30 causes a response in a thin film electroactive microactuator 32. Lateral strain in the thin film electroactive microactuator 32 causes a flexible membrane 34 to exert force upon the neurotransmitter chemical fluid, as seen in FIG. 6B. One or more outlets 36, e.g., microvalves, microchannels or microports provide outlets for dispensing fluid. When irradiated by light, the flexible membrane/diaphragm actuator provides pressure to dispense neurotransmitter chemical fluid through the outlets 36. The amount of fluid ejected and the rate of ejection are regulated by the intensity and the irradiation time/frequency of the incident light. The outlets 36 are formed in a separate wafer, which is micromachined separately and then bonded to the wafer containing the integarated solar cell, flexible membrane 34, and electroactive thin film actuator 32 through fusion bonding or another suitable technique.

Design Criteria

Available light for actuation and the required actuation force can guide the specific design criteria for a given implementation of a light powered microactuator of the invention, for example a retinal prosthesis or another microfluidic pump. The following discussion will guide artisans seeking to achieve specific operational values in specific environments.

The retinal prosthesis of FIGS. 6A and 6B will be used to consider a specific implementation, which may include an array of microactuators in accordance with FIGS. 6A and 6B. Guided by application constraints, the height is limited to about 0.5 mm, and the overall lateral dimensions of a single microfluidic pump are based on the number of the microdispenser units considered in the prosthesis array, whose size must be limited to 1 cm×1 cm. The design of microdispenser units for two array sizes, a 5×5 array and a 10×10 array, are considered here. Accordingly, the overall lateral dimensions of the microdispenser corresponding to these two array sizes are set to be 2 mm×2 mm and 1 mm×1 mm respectively.

For the design guidance considered here, the electroactive (PZT) layer and a passive silicon membrane layer are considered at the center of the dispenser unit and the area surrounding the microactuator comprises the solar cell. The integrated solar cell is connected across the electroactive microactuator via electrodes on either side of the electroactive layer. The micro/nano-scale outlet ports are essentially microchannels of diameters of about 0.5-3 μm through the flat surface of the chamber structure.

The solar cell is assumed to be formed in an square area (having sides of length l) bounded between the outer edges of the dispenser (L) and the perimeter of a circular microactuator membrane (where l=1.8 mm and L=2 mm for the 5×5 array configuration and l=0.9 mm and L=1 mm for the 10×10 array configuration). A solar cell's performance is typically characterized by its open-circuit voltage, closed circuit current density, fill factor and efficiency determined via experimentally obtained J-V (current density vs. voltage) curve, measured under standard AM1.5 conditions, i.e. a temperature of 25° C. and a solar spectrum irradiance, I₀=1000 W/m². The current density (current per unit area of the solar cell) and voltage are related through a non-linear function, and their values at any instant during the operation of the cell depend on the magnitude of the load resistance across the cell at the instant.

The short circuit current density of a solar cell (under standard AM1.5 conditions), which varies linearly with change in illumination relative to the standard values, is given by:

$${J}_{s c} ( I ) = I \frac{{J}_{s c}^{0}}{{I}_{0}} \qquad ( 1 )$$

and the open circuit voltage, which varies logarithmically, is given by:

$${V}_{o c} ( I ) = \frac{k T}{{e}_{0}} ln ( \frac{{J}_{s c}}{{J}_{0}} + 1 ) = \frac{k T}{{e}_{0}} ln ( \frac{I}{{I}_{0}} \frac{{J}_{s c}^{0}}{{J}_{0}} + 1 ) \qquad ( 2 )$$

where, I is the given irradiance, J_{sc}⁰ is the measured short circuit current at the standard irradiance of I₀, J₀ is the dark current density, k is Boltzmann's constant, T is the solar cell temperature in Kelvin, and e₀ is unit electron charge.

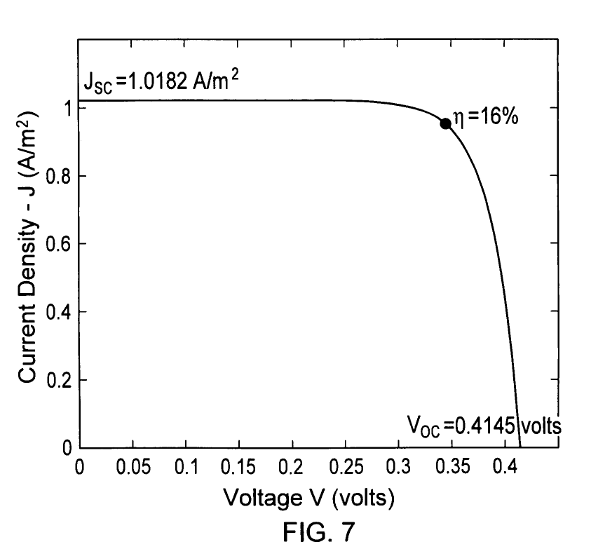

For the solar cell design in the retinal prosthesis microactuator application, a solar cell with the following average performance characteristics is considered: efficiency η_{cell}=16% and short circuit current density J_{sc}⁰=33.94 mA/cm², both characterized at 1000 W/m². Furthermore, the following characteristic values for a crystalline silicon are assumed: J₀=1×10⁻¹² A/cm² and T=298 K (25° C. room temperature). The standard J-V curve of the solar cell corresponding to the assumed set of characteristic values and an irradiance of 3 W/m² is plotted in FIG. 7. The J-V curve plot shows that the solar cell generates an open circuit voltage V_{oc}=0.4145 volts and a close circuit current density J_{sc}=1.0182 A/m². The J-V curve plot also shows the operating point on the curve corresponding to the maximum rated efficiency of the cell η_{cell}=16%. At any other operating point on the curve, the efficiency of the cell will be less than the rated maximum efficiency and determined by the magnitude of electrical load connected across the cell.

When this solar cell area is irradiated by light, the cell produces an open circuit voltage as a function of the intensity of the light and serves as a voltage source across the electroactive thin film actuator. However, unlike an ideal voltage source (or a battery), the voltage supplied by the solar cell at the given intensity of irradiation varies from 0 to the maximum open circuit voltage, V_{oc}, depending on the load connected across the cell.

Electroactive Microactuator Modeling

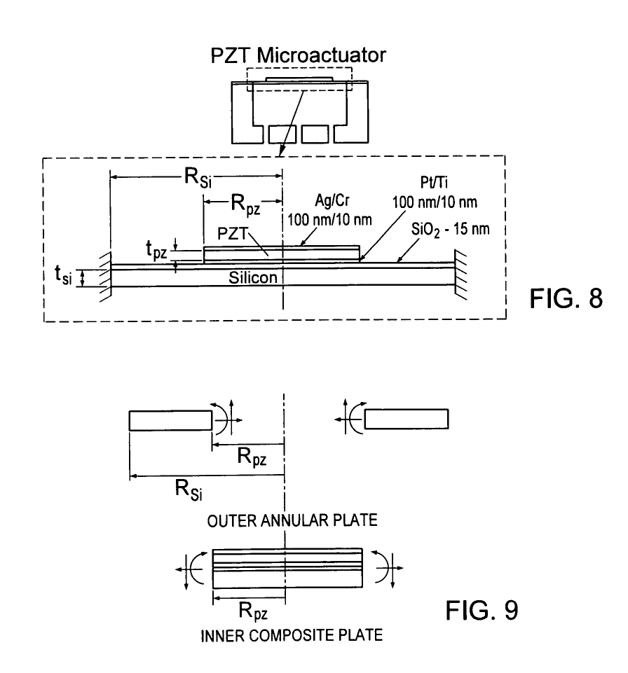

A 2D schematic of the microactuator along with its major design variables and the electrodes on both sides of the electroactive layer is illustrated in FIG. 8. The electrodes comprised of thin layers of Ag/Cr and Pt/Ti on the top and bottom sides of the electroactive layer respectively will have the same radii as the electroactive layer and fixed thickness as shown in FIG. 8. This choice of electrode materials is dictated by their compatibility with the thin-film microactuator fabrication process. Additionally, a thin layer of SiO₂ exists on top of the silicon layer to serve as a diffusion barrier between the bottom Pt/Ti layer and the bottom silicon layer. The four key dimensions of the microactuator that will be optimized for the desired microactuator performance are the radii and thickness of the silicon and electroactive layers, viz. Rₛᵢ, R_{pz}, tₛᵢ, t_{pz}, as shown in FIG. 8.

The microactuator motion is effected by two opposing forces: the downward force effected by electroactive material when voltage, V, is applied across the layer and an upward pressure, P, effected by the liquid in the dispenser chamber. An expression for the net volumetric displacement of the microactuator under combined loading of the electrical field and the pressure, needed for the optimization of its dimensions in following section, is derived below.

The derivation approach utilizes Kirchoff's plate theory for a composite structure applied separately for the inner composite extending from the center up to the edge of the electroactive layer and the outer annular composite extending from the edge of the electroactive layer to the outer boundary of the silicon membrane. Following the Kirchoff's plate theory for bending of an axi-symmetrically loaded circular composite plate, it can be shown that the general solutions for transverse deflection, w(r), and lateral deflection, u(r), in the mid-plane of a composite plate in FIG. 8 under a pressure loading, P, on one side and an electrical voltage, V, across the electroactive thin film

$$\begin{matrix}w ( r ) = \frac{P {r}^{4}}{64 ( {D}_{11} - \frac{{B}_{11}^{2}}{{A}_{11}} )} + {c }_{1} {r}^{2} + {c}_{2} ln ( r ) + {c}_{3} {r}^{2} ln ( r ) + {c}_{4} & ( 3 ) \\ u ( r ) = \frac{{B}_{11}}{16 {A}_{11}} \frac{P {r}^{3}}{( {D}_{11} - \frac{{B}_{11}^{2}}{{A}_{11}} )} + {c}_{3} \frac{{B}_{11}}{{A}_{11}} r ( 2 ln ( r ) - 1 ) + {c}_{5} r + \frac{{c}_{6}}{r} & ( 4 )\end{matrix}$$

cᵢ (i=1 to 6) in equations (3) and (4) are six constants that can be evaluated through appropriate boundary conditions. Although the applied electrical voltage load, V, does not explicitly appear in these equations, it, however, influences the displacement solution through the constants as boundary conditions. The electroactive force and moment terms are also included in the solution through the boundary conditions. The terms A₁₁, B₁₁, and D₁₁ are calculated from the equations below,

$$matrix [ \begin{matrix}{A}_{11} & {A}_{12} \\ {A}_{12} & {A}_{11}\end{matrix} ] = {\sum }_{i = 1}^{n} {\int }_{{z}_{i - 1}}^{{z}_{i}} \frac{{E}_{i}}{1 - {v}_{i}^{2}} [ \begin{matrix}1 & {v}_{i} \\ {v}_{i} & 1\end{matrix} ] d z \text{-} Extensional stiffness \\ matrix [ \begin{matrix}{B}_{11} & {B}_{12} \\ {B}_{12} & {B}_{11}\end{matrix} ] = {\sum }_{i = 1}^{n} {\int }_{{z}_{i - 1}}^{{z}_{i}} \frac{{E}_{i}}{1 - {v}_{i}^{2}} [ \begin{matrix}1 & {v}_{i} \\ {v}_{i} & 1\end{matrix} ] z d z \text{-} Flexure stiffness \\ matrix [ \begin{matrix}{D}_{11} & {D}_{12} \\ {D}_{12} & {D}_{11}\end{matrix} ] = {\sum }_{i = 1}^{n} {\int }_{{z}_{i - 1}}^{{z}_{i}} \frac{{E}_{i}}{1 - {v}_{i}^{2}} [ \begin{matrix}1 & {v}_{i} \\ {v}_{i} & 1\end{matrix} ] {z}^{2} d z \text{-} Bending stiffness \qquad ( 5 )$$

where, Eᵢ is the Young's modulus, vᵢ is the poisson's ratio for each i^{th} layer, and the properties are summed over n layers. zᵢ₋₁ and zᵢ are the heights of the bottom and the top respectively of the iₜₕ layer from an arbitrarily chosen mid-plane.

In order to analyze a composite plate with layers of unequal radii, the microactuator is treated as a superposition of two plates: an outer annular plate and an inner composite plate as schematically illustrated in FIG. 9. Equations (3) and (4) are applied separately for the two plates, and the 12 constants for the four equations are solved for with appropriate boundary and interface conditions as follows:
1. Clamped outer annular plate (3 equations)
2. Bounded solution at the center of the plate (3 equations)
3. Continuity of slopes, and equal transverse and lateral displacements at the interface (3 equations)
4. Compatibility of lateral force, moment, and shear force at the interface (3 equations). The electrical voltage term appears in the displacement solution through this boundary condition.
   The net volume displaced by the microactuator, v, can be determined by the area integration of the transverse displacement solutions, obtained as two separate expressions for the inner composite plate and outer annular plate (denoted here as w_{c}(r) and wₐ(r)):

$$v = {\int }_{0}^{a} 2 π r {w}_{c} ( r ) d r + {\int }_{a}^{b} 2 π r {w}_{a} ( r ) d r \qquad ( 6 )$$

After evaluating the integrations in equation (6), the resulting expression for the net volume displaced by the microactuator can be expressed in a convenient form as the net difference of the volume changes effected by the electric field across the piezo layer and the fluid pressure in the dispenser chamber due to the microactuator displacement:

v=C_{V}V−C_{P}P (7)
where V is the applied voltage across the piezo layer, P is the pressure generated in the dispenser chamber, CV and CP are the volumetric compliances of the microactuator defined as:

$$\begin{matrix}{C}_{V} = \frac{v}{V} {\vert}_{P = 0} = Voltage \text{-} induced volumetric compliance & ( 8 a ) \\ {C}_{P} = \frac{v}{P} {\vert}_{V = 0} = Voltage \text{-} induced volumetric compliance & ( 8 b )\end{matrix}$$

C_{V} and C_{P} are functions of the microactuator's dimensions (see FIG. 7) and its material properties, viz., Young's modulus E, Poisson's ratio, v, and the electroactive constant, d₃₁ (The full expressions for C_{E} and C_{P}, which were obtained using Maple, are not reported here due to their somewhat unwieldy size):

C_{V}≡C_{V}(Rₛᵢ, R_{pz}, tₛᵢ, t_{pz}, Eₛᵢ, vₛᵢ, E_{pz}, v_{pz}, d₃₁) (9a)

C_{P}≡C_{P}(Rₛᵢ, tₛᵢ, Eₛᵢ, vₛᵢ, R_{pz}) (9b)

Tailoring microactuator performance for dispensing the liquid in the dispenser chamber through each of the outlet ports 36 of FIGS. 6A and 6B at an average rate of about 1 pl/s when the power density of the input light irradiance is 3 W/m² is a well-suited design goal for an exemplary embodiment retinal prosthesis. The chief performance characteristics of the microactuator that influence the desired input-output requirements of the microdispenser are: the total volume displaced by the microactuator, the rate of volume displacement by the microactuator, and the mechanical strength of the microactuator for a reliable long-term operation. Of these, the rate of displacement, as well as the voltage supplied to the microactuator, are governed by the solar cell characteristics and the light collection area of the cell. In this design, since the solar cell region is rather limited (bounded by the outer edges of the device and the perimeter of the membrane), the solar cell design is treated independently, and as such, the rate of volume displacement by the microactuator is not explicitly considered in the microactuator design. The other characteristics of the microactuator performance, viz. the volume displaced and the microactuator material strength, are tailored through the four key dimensions of the microactuator.

The microactuator design, thus, entails determination of the optimal thickness and radii of electroactive and silicon layers, which is accomplished via the following criteria: the volume displaced by the microactuator is maximized for a constant voltage applied across the electroactive layer while the maximum stress in the critical regions of the microactuator is limited to be well below failure limits. Maximization of the microactuator volume displacement, within the design bounds, is useful because a larger microactuator volume displacement would allow for a higher density of outlet ports per dispenser unit with each outlet port dispensing liquid at the same desired rate, which is desirable from the dispenser's application perspective.

Thin-film piezoelectric actuators fail mainly due to cracking of the piezoelectric film, followed by delamination of the piezo layer and the bottom electrode layer. Therefore, of all the layers of various materials in the multi-layer microactuator structure considered, the electroactive layer is most susceptible to failure, and as such, it is important to consider the operational peak stress in the electroactive layer during the microactuator design. Another important aspect to consider in the microactuator design in regards to the peak operational stresses in the electroactive layer is a tensile residual stress that is inevitably introduced during the fabrication process. Experiments involving four-point bending tests on sol-gel deposited 53/47 PZT thin films have shown that cracking occurs in the PZT material occurs at 198.14 MPa, of which 117.21 MPa is the tensile residual stress and that thin-film PZT material can withstand a maximum operational tensile stress of 80.93±16.22 MPa on top of the residual stress. Although this reported failure stress value is specific to the PZT composition, its processing conditions and the physical geometric constraints of the test samples, it is, nevertheless, useful as a guiding upper limit stress, especially when applied in conjunction with an appropriate factor of safety. Consequently, for a conservative design, the lower value of the tensile stress limit reported, i.e. 64.71 MPa (80.93−16.22 MPa) is adopted as the limiting upper design value of the stress in the thin-film electroactive microactuator, and in addition, to take into account any unpredictable post-fabrication residual stress, the design stress limit is further reduced by a factor of safety of 5. That is, the peak stress in the electroactive material, σ_{pz}^{max}, is limited to 12.94 MPa.

With the above considerations, the design optimization problem is posed as follows:

Max(v) (10)
subject to:
   Equilibrium equations, and
Boundary conditions:

Rₛᵢ^{man}<Rₛᵢ<Rₛᵢ^{max}

R_{pz}^{min}<R_{pz}<R_{pz}^{max}

tₛᵢ^{min}<t_{pz}<tₛᵢ^{max}

t_{pz}^{min}<t_{pz}<t_{pz}^{max}

σ_{pz}^{max}≦12.94 MPa

V_{oc}=0.4145 V

The objective function, v, is given by the expression in Eq. (7) when P=0, without any loss of generality. Furthermore, in this objective function, the voltage induced compliance, C_{V}, a function of various design variables as expressed by Eq. 9(a) and related to the mechanics of equilibrium as expressed by Eqs. (3) through (5), is derived considering only four layers silicon (Si), platinum (Pt), piezoelectric (PZT), and silver (Ag) layers. The SiO₂, Ti, and Cr layers, whose thickness is only about 10-15 nm, are not considered in the optimization since they are structurally insignificant relative to other layers. The open circuit voltage value applied in the optimization, V_{oc}=0.4145 volts, corresponds to an illumination intensity of 3 W/m².

The maximum and minimum values of the four major dimensions, applied in the optimization for the two array sizes considered, are listed in Table 1.

TABLE 1
Limiting values of the design variables in the design optimization (Eq. (10)).

| Array | Rₛᵢ | | R_{pz} | | tₛᵢ | | t_{pz} | |
|---|---|---|---|---|---|---|---|---|
| size | Rₛᵢ^{min} | Rₛᵢ^{max} | R_{pz}^{min} | R_{pz}^{max} | tₛᵢ^{min} | tₛᵢ^{max} | t_{pz}^{min} | t_{pz}^{max} |
| 5 × 5 | 100 μm | 800 μm | 50 μm | 800 μm | 10 μm | 30 μm | 0.1 μm | 3 μm |
| 10 × 10 | 100 μm | 400 μm | 50 μm | 400 μm | 5 μm | 20 μm | 0.1 μm | 3 μm |

The four materials involved in the exemplary design, Si, Ag, Pt, and PZT, exist as thin-films, and hence, appropriate thin-film properties must be applied in the design. The thin-film properties of interest in the design are the elastic modulus, E, and the poisson's ratio, v, for all the four materials, and the transverse piezoelectric coefficient, d₃₁, for PZT. While these thin-film properties of Si, Ag, and Pt are well characterized, the thin-film PZT properties, especially its d₃₁ coefficient, are not. For the microactuator design optimization, the experimentally determined values published for the elastic modulus and poission's ratio for all the materials, which are summarized in Table 2, are adopted.

TABLE 2
Thin-film properties of the materials applied in the
microactuator design.

| Property | Si | Pt | Ag | PZT |
|---|---|---|---|---|
| Young's | 168.9 [23, 24] | 170 [25] | 82.7 [25] | 120 [26, 27] |
| modulus, E (GPa) | | | | |
| Poisson's ratio, ν | 0.182 [24] | 0.38 | 0.37 | 0.45 [27] |

The transverse coefficient d₃₁ value of a thin film PZT material, unlike bulk PZT, is strongly influenced by the mechanical clamping on the substrate and the electrode since the PZT film is a part of the composite film-substrate structure. Therefore, the effective value of the d₃₁ coefficient cannot be measured directly and is typically estimated indirectly by measuring the in-plane stress in the PZT film and relating it to the d₃₁ coefficient through the relation:

$${e}_{31 f} = \frac{{d}_{31}}{{S}_{11}^{E} + {S}_{12}^{E}} = \frac{{d}_{31} {E}_{pz}}{( 1 + v )} \qquad ( 11 )$$

where e_{31f} is the thin-film piezoelectric constant (in-plane stress per unit electric field), and S₁₁^{E} and S₁₂^{E} are mechanical compliances of the PZT film. Thus, the estimation of transverse piezoelectric coefficient d₃₁ requires explicit knowledge of the elastic modulus of the PZT thin-film, which is not well characterized and has values ranging from 37 GPa to 400 GPa reported in the literature. Consequently, there are wide disparities among d₃₁ values reported in the literature, which could be attributed to the use of different elastic moduli for PZT in their calculation.

Therefore, for the exemplary retinal prosthesis microactuator design, the d₃₁ value is calculated using the relation in Eq. (11) and a published value for E_{pz} that is experimentally determined (indicated in the last column of Table 2). The thin-film piezoelectric constant e_{31f} values range from −8 to −12 C/m². Corresponding to these e_{31f} values, the effective thin-film transverse coefficient, d₃₁ is calculated using Eq. (11) to range between −96.67 pC/N and −145 pC/N. For the current design purposes, a value of d₃₁=−100 pC/N, which is on the lower end of the range calculated and consistent with some of the published values for d₃₁ for PZT thin-films, is adopted.

The optimization problem was implemented and solved in the optimization toolbox of Matlab® using the fminimax routine. The optimized dimensions and the corresponding volume and maximum stress in the piezoelectric layer for the two different sizes of the dispenser unit corresponding to 10×10 and 5×5 array configurations, are summarized in the following table. For convenience of reference, the designs corresponding to the 10×10 and 5×5 array configurations will henceforth be called as Design A and Design B, respectively.

TABLE 3
Results of the design optimization for the two design sizes.

| | Array | Rₛᵢ | tₛᵢ | R_{pz} | t_{pz} | ν_{disp} | σ_{pz} |
|---|---|---|---|---|---|---|---|
| | Size | (μm) | (μm) | (μm) | (μm) | (pl) | (MPa) |
| Design A | 10 × 10 | 400 | 5 | 296 | 0.61 | 29.38 pl | 12.9 |
| Design B | 5 × 5 | 800 | 10 | 580 | 0.65 | 121.64 pl | 12.9 |

The analytical model set forth was confirmed with finite element analysis. The material layers, the dimensions, material properties and boundary conditions applied in the finite element model were consistent with the corresponding values in the optimized design for the Design A. The finite element analysis showed that under a voltage load of V=0.4145 volts, the resultant maximum stress in the piezoelectric layer away from the interface was found to be consistent with the predicted value of 12.94 MPa for the PZT.

A comparison of the displacements at points along a diameter of the microactuator under various loading conditions obtained both analytically by Eqs. (3) and (4) and through the finite element analysis was also conducted.

The results for the following loading conditions on the microactuator are compared: Two cases of only voltage load (0.33 and 0.42 V) that causes a downward deflection of the microactuator, two cases of only pressure load (0.5 kPa and 1 kPa) that causes an upward deflection of the microactuator, and one case of combined voltage and resistive pressure load (0.42 V and 0.8 kPa). The analytical and finite element analysis results are found to be in excellent agreement (within an error of 0.5% for pressure and voltage loading cases, and within an error of 3.5% for the combined loading case) thereby validating the analytical model used in the design optimization.

Response Time

The microactuator's response time is influenced by the solar cell area available for collection of light. In general, the larger the solar cell collection area, the faster the microactuator response. However, for the solar cell area considered, the microactuator response time is on the order of a few milliseconds, and hence, for a bench-top prototype, the resulting response time is satisfactory for a retinal prosthesis device, even with a further reduction in the solar cell collection area.

Example Prototype Devices Fabrication Method and Device Characterization

### Device Layout

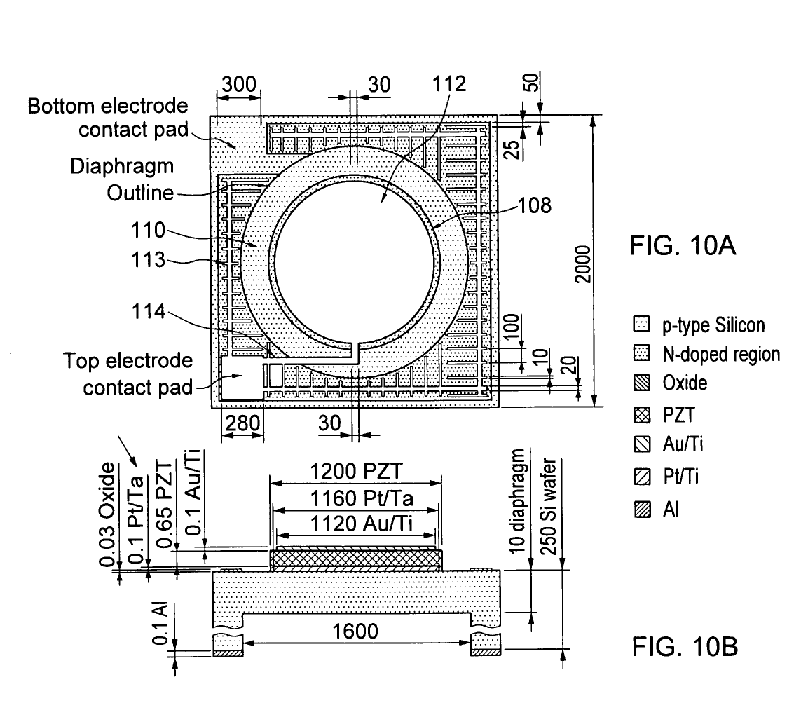

A 2D schematic of the example prototype integrated microactuator device is shown in FIGS. 10(A) and 10(B) along with its dimensions. The device comprises a circular, unimorph type PZT thin film microactuator located at the center and a crystalline silicon solar cell around the periphery of the actuator on the same silicon die. The microactuator is a multi-layer structure comprised of a circular silicon diaphragm and a circular PZT thin film layer 108 sandwiched between a bottom electrode layer 110 (Pt/Ta) and a top electrode layer (Au/Ti) 112. A solar cell collection grid 113 (or the top electrode for the solar cell) is connected to the top electrode layer of the PZT layer via a metal bridge 114 and both are of the same material. The bottom electrode layer of the solar cell (Al) is on the backside of the silicon diaphragm. It is connected to the bottom electrode of the PZT and grounded to complete the electrical circuit.

The optimal dimensions of the microactuator were derived using an analytical model for the integrated behavior of the solar cell and the circular piezoelectric microactuator. See, M. Deshpande and L. Saggere, “Modeling and Design of an Optically Powered Microactuator for a Microfluidic Dispenser,” J Mechanical Design, 127(4), pp. 825-836, July 2005. The dimensions were optimized for maximizing the actuator deflections under low intensity light of 3 W/m² for bench top testing purposes. As seen in FIG. 10A, the overall footprint area of the device is 2×2 mm² and its thickness is less than 0.25 mm. The silicon diaphragm is 1600 μm in diameter and 10 μm in thickness. The PZT layer is 1580 μm in diameter with a thickness of 650 nm, deposited over a 100 nm thick Pt/Ta bottom electrode layer. The solar cell area, located at the periphery of the microactuator has an overall area of 1.448 mm². A collection grid is includes several 10 μm wide collection fingers and 20 μm bus-bars. The grid and the top of electrode of the PZT layer are formed from a 100 nm thick Au/Ti layer. This design includes the following two features to facilitate and simplify the integration of the solar cell and piezoelectric microactuator components: i) a single oxide layer is used to serve as both a diffusion barrier layer required in deposition of PZT on silicon and a surface passivation layer for the solar cell to reduce surface recombination losses, and ii) the PZT layer is extended beyond its two electrode layers by about 10 μm to ensure that the two electrodes will remain insulated even in case of small misalignment of the layers or over etching of the PZT thin film layer.

### Device Fabrication

The microactuator was fabricated on a p-type 4-inch <100> oriented silicon wafer. First, a 750 nm-thick layer of wet oxide was thermally grown at 1100° C. and patterned to form the diffusion mask for fabrication of the solar cell emitter region. A spin-on-phosphorus dopant source (Filmtronics Inc., P509) was deposited over the patterned oxide and baked for 1 hr at 200° C. to remove the solvents.

Following the bake, the diffusion was carried out in a tube furnace at 1000° C. for 30 minutes in pure oxygen environment followed by the stripping of the by-products consisting mostly of oxide layers in 49% HF solution. Next, a 40 nm thick layer of dry oxide was grown on the wafer at 1000° C. for 30 minutes, which serves as both a diffusion barrier layer for the PZT material and as the solar cell passivation layer.

Following the solar cell formation, a 100/10 nm thick Pt/Ta bottom electrode layer was deposited using e-beam evaporation and patterned using the lift-off technique to form the bottom electrode layer for the PZT thin film. Then, approximately 650 nm-thick film of PZT (composition 115/52/48 from Mitsubishi Electronic Materials Inc.) was deposited on top of the bottom electrode via the sol-gel approach. Each layer accounting for approximately 40 nm was spread at 600 rpm for 3 s, spun at 3000 rpm for 20 s and pyrolized at 375° C. in air for 5 minutes. To minimize the stress buildup during this process, the PZT film was patterned after every 5-6 sol-gel layers in 2:1 HCl:DI mixture at 55° C. with 1% HF using a photoresist mask. The patterned film was then crystallized at 650° C. for 90 s. The deposition-patterning-annealing steps were repeated to obtain an approximate PZT thickness of 650 nm.

Following the deposition of the PZT film, the oxide layer over the solar cells area was patterned in 10:1 BOE. Next, a layer of 100/10 nm thick Au/Ti metal films were deposited via e-beam evaporation and patterned using the lift-off technique to create the solar cell collection grid as well as the top electrode of the solar cell. Following this, the backside electrode for the solar cell comprising 100 nm of Al is deposited via e-beam evaporation.

A diaphragm was then fabricated through the backside etch of the wafer using DRIE (Deep Reactive Ion Etching). For this etching, the Al film on the backside was patterned and then the wafer was etched in an Oxford Plasmalab 100 system using a thick (˜18 μm) photoresist film (SPR-2207.0 μm, Rohm and Haas materials) as masking material for the DRIE process. The etch depth was monitored at several intervals to obtain diaphragms of ˜10 μm thickness. Finally, the photoresist was stripped away and test devices were diced to complete the fabrication process.

A 2×2 mm² prototype was used for testing. The various regions, such as the solar cell collection area, the collection grid, the PZT thin film layer and contact pads were consistent with FIGS. 10A and 10B.

### Device Performance Characterization

For the test and characterization of the optically powered microactuator three test prototypes were selected that were identical in all respects except the thicknesses of their silicon diaphragms. The thicknesses of the diaphragms of the three prototypes, labeled as prototype 1, prototype 2 and prototype 3, were 9.1 μm, 13.2 μm and 14.63 μm, respectively. This variation in thickness was due to the variations in the DRIE process across the wafer. Various experiments were carried out to characterize the mechanical and electrical output parameters of the microactuator. These included the measurement of the voltage-induced displacement of the PZT microactuator component on the integrated devices, the solar cell photovoltage component, and the optically powered displacements of the integrated microactuator. Additional experiments were conducted on prototype 2 in order to model the integrated microactuator and characterize its temporal behavior along with the mechanical output parameters such as volume displacement, block pressure and mechanical power output.

Experimental Setup

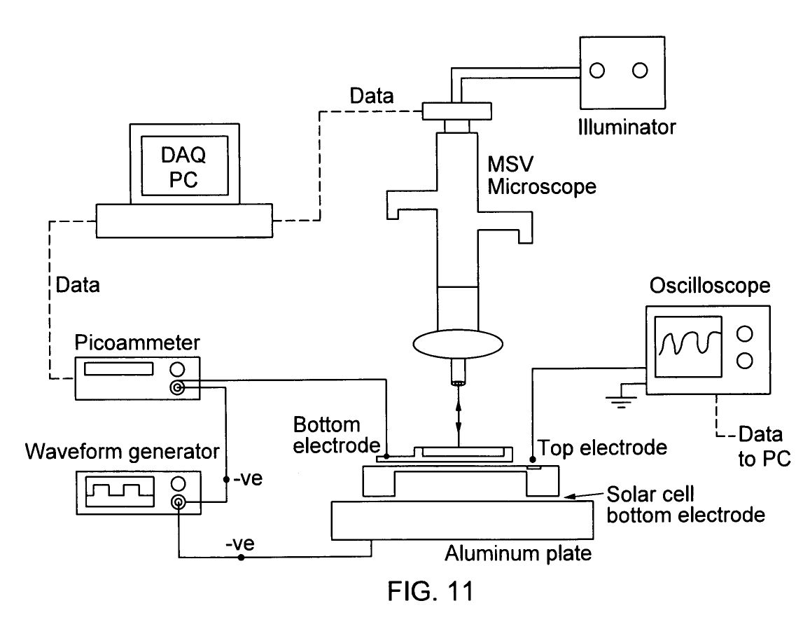

The experimental setup for the characterization of the integrated optically powered microactuator is shown in FIG. 11. The major instrumentation comprised a Microscope scanning vibrometer (MSV-400 with displacement decoder DD-200, Polytec Inc.) for displacement measurement, a waveform generator (Agilent 33220A) for voltage excitation, a two-channel oscilloscope (TDS 2012 Tekronix) and a Picoammeter (6487 Keithley) for current voltage measurement, a microscope illuminator (Nikon) equipped with a heat-blocking filter for illumination and an optical power meter for calibration of the illuminator (841-PE with 818SL-ST detector, Newport). The measured data was logged into a PC via GPIB using LabVIEW® software. The light related experiments were carried out in a dark room to eliminate the effect of ambient lighting. The PZT layer on the device prototypes was poled at 11 V (electric field of about 170 KV/cm) prior to their testing.

### Test Results

The device characterization was carried first at the component level viz., the PZT actuator and the solar cell were tested independently, and then at the system level, i.e., the integrated solar cell and PZT actuator.

Testing of the Piezoelectric Actuator

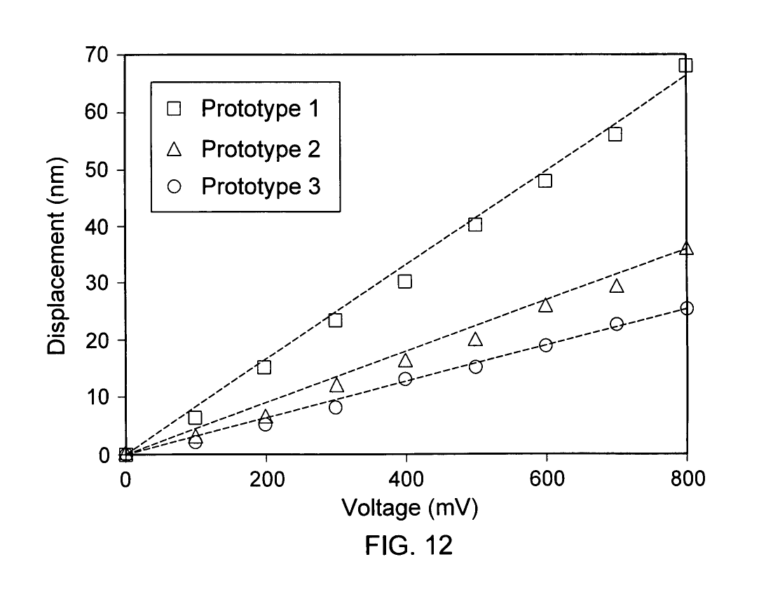

To verify the characteristic of the PZT thin film microactuator component alone on the integrated device, the microactuator was actuated using voltage externally supplied by the waveform generator connected to the top and bottom electrodes of the PZT film via probes. The center point displacements were measured for 100 Hz unipolar sinusoidal excitation signal of varying magnitudes in the range 0-700 mV in steps of 100 mV. The center deflections of the three actuators, measured as a function of externally applied voltage, are plotted in FIG. 12. As can be seen in the plot, a thicker diaphragm deflects less than a thinner diaphragm due to its higher structural stiffness as expected. At 700 mV of input voltage, prototype 1, with 9.1 μm thick diaphragm (thinnest) produced the largest deflection of 56 nm, while prototype 3 with a diaphragm thickness of 14.63 μm (thickest) produced the lowest deflection of 22.6 nm.

Testing of the Solar Cell

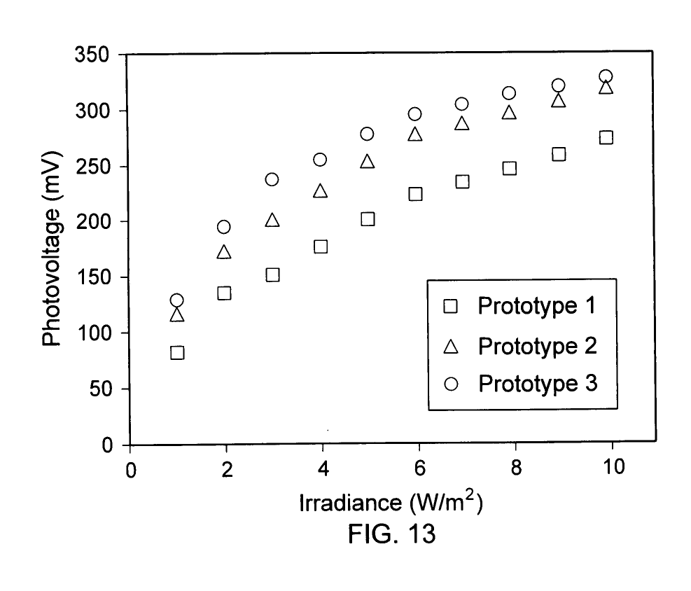

To verify the characteristic of the solar cell component alone on the integrated device, prototypes were irradiated with a white light from the Nikon microscope illuminator that was calibrated for an output from 1-10 W/m² with using the Newport 841-PE optical power meter set at 595 nm wavelength. The photovoltage generated by the solar cell was measured by means of an oscilloscope connected to the solar cell electrodes via probes. The open circuit voltages, V_{oc}, measured across the solar cells on the three prototypes are plotted as functions of irradiance in FIG. 13. It can be seen that a maximum of 327 mV voltage is generated by prototype 3 at an irradiance of 10 W/m². Small variations noted in the photovoltages generated by the three prototypes in FIG. 13 could be attributed to the microfabrication process tolerances on a wafer.

Testing of the Integrated Solar Cell and the PZT Actuator Device

Following the component testing, the performance of the integrated microactuator was characterized by measuring the actuator deflections as functions of irradiated light intensities in the range 0-10 W/m². A calibrated white light from Nikon microscope illuminator was used to excite the solar cell region of the device. To enable the deflection measurements using the micro scanning vibrometer (utilized for its high resolution measurement capability) that is capable of measuring only dynamic displacements, the photovoltage output from the solar cell (input power to the actuator) was modulated by a 100 Hz sinusoidal bias signal of peak amplitude equal to the solar cell's open-circuit voltage from a signal generator. The oscilloscope connected to the top electrode contact pad was used to measure and monitor the photovoltage during the modulation. Using this technique, the prototypes were excited at irradiances between 1-10 W/m² in steps of 1 W/m².

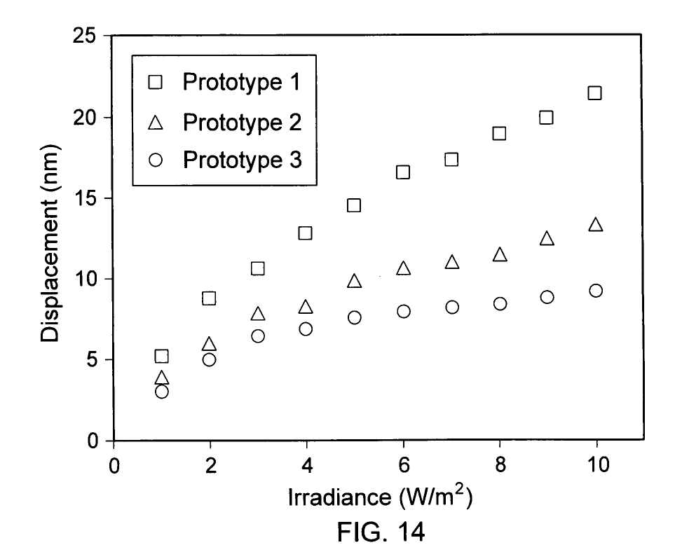

The deflection-irradiance characteristics of the three prototype devices are plotted in FIG. 14. The maximum deflections of prototypes 1, 2 and 3 were 21.4 nm, 13.4 nm and 9.2 nm respectively for an incident irradiance of 10 W/m².

Model-Experiment Validation

The experimentally characterized results of the optically powered microactuator device were validated by analytical models—first at the component level and then at the integrated system level.

Validation of Solar Cell Characteristic

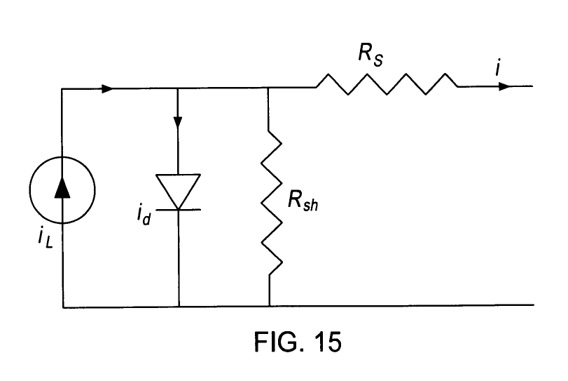

The solar cell component was modeled using a single-diode model for a non-ideal solar cell that includes the effect of the solar cell resistances and a non-ideal diode behavior. This model, illustrated in FIG. 15, can be analytically expressed as,

$$I ( G ) = {I}_{L} ( G ) - {I}_{0} ( {e}^{\frac{V ( G ) + I {R}_{s}}{n {V}_{T}}} - 1 ) - \frac{( V ( G ) + I ( G ) {R}_{s} )}{{R}_{sh}} \qquad ( 12 )$$

where V is the voltage, I is the current, I_{L} is the photocurrent, I₀ is the reverse saturation current, n is known as the ideality factor or diodicity, V_{T} is a constant given as V_{T}=0.0257 V at 25° C., Rₛ is the series resistance of the cell, Rₛₕ is the shunt resistance of the solar cell. Of these parameters, I, I_{L} and V are dependent on the irradiance G and the other values are relatively constant for a given solar cell.

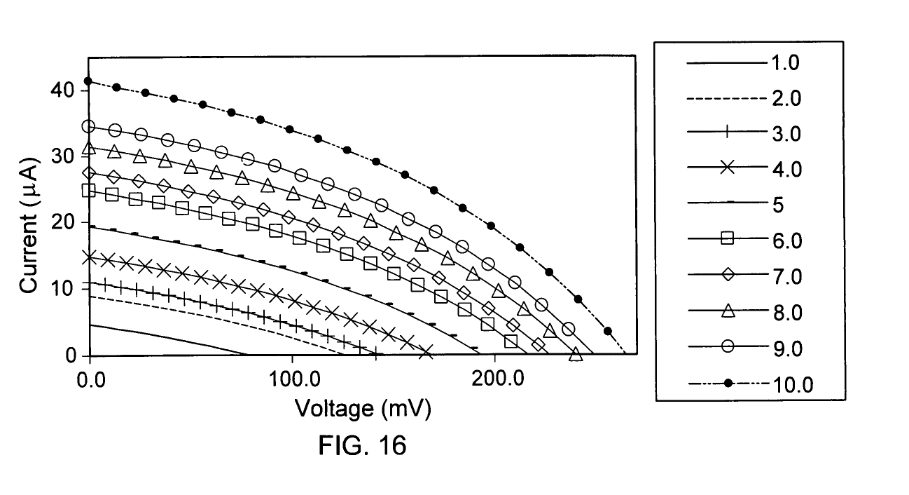

The I-V curves of the solar cell in prototype 2 were experimentally measured for irradiances in the intensity range 1-10 W/m² and plotted (FIG. 16) using a picoammeter with a built in voltage source. Using these I-V curves and a curve fitting software for solar cell modeling called IVFIT, the parameters I_{L}, I₀, Rₛ, Rₛₕ and n were extracted for various irradiance values, and average values of the irradiance independent parameters, I₀*, Rₛ*, Rₛₕ* and n*, over the entire intensity range were computed (see Table. I). The photogenerated current I_{L}, which is known to be directly proportional to irradiance, was normalized with the irradiance and an average was taken over the measurements to calculate a key modeling parameter denoted as, i_{L}. This, in terms of i_{L}, the photogenerated current can be expressed as I_{L}(G)=i_{L}G. Besides these parameters, the solar cell fill factor, FF, the current, Iₘ, and voltage, Vₘ, at the maximum power point, Pₘ, were also estimated. Using the above parameters, a generic single-diode model is obtained as,

$$I ( G ) = {i}_{L} ( G ) - {I}_{0}^{*} ( {e}^{\frac{V ( G ) + I {R}_{s}^{*}}{{n}^{*} {V}_{T}}} - 1 ) - \frac{( V ( G ) + I ( G ) {R}_{s}^{*} )}{{R}_{sh}^{*}} \qquad ( 13 )$$

In order to validate the model given by Eq. (13), analytically obtained values of the short-circuit current, I_{sc}, and the open circuit voltage, V_{oc}, were compared with the corresponding values measured experimentally. The expressions for the short circuit current (the current generated by the solar cell when the voltage is zero) I_{sc}, and the open circuit voltage (the voltage generated by the solar cell when the current is zero) V_{oc}, obtained from Eq. (2) are:

$$\begin{matrix}{I}_{sc} = {i}_{L} G \frac{{R}_{s}^{*}}{{R}_{s}^{*} + {R}_{sh}^{*}} & ( 14 ) \\ 0 = {i}_{L} G - {I}_{0}^{*} ( {e}^{\frac{{V}_{o c} ( G )}{{n}^{*} {V}_{T}}} - 1 ) - \frac{{V}_{oc} ( G )}{{R}_{sh}^{*}} & ( 15 )\end{matrix}$$

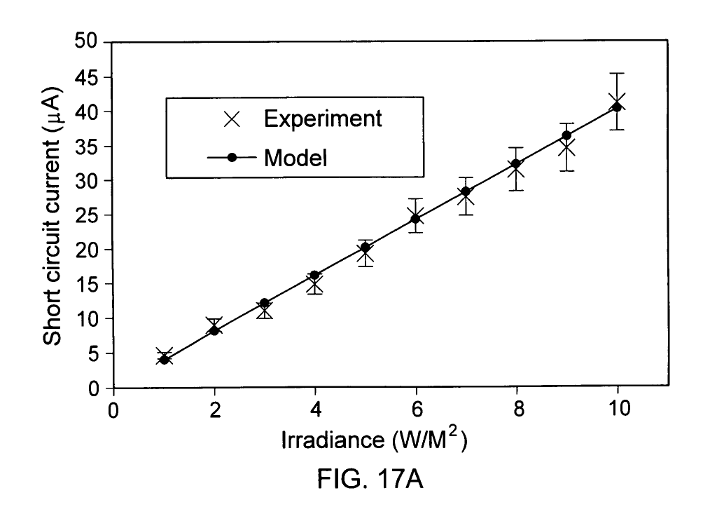

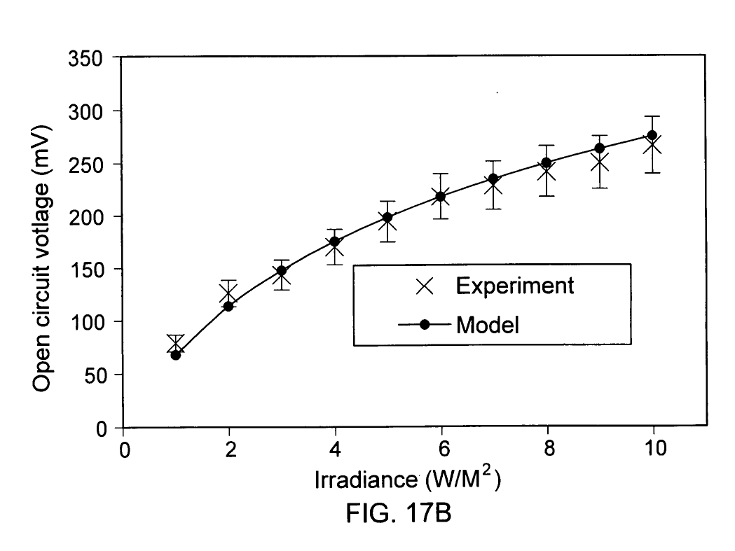

The experimental and analytical values for I_{sc} are plotted in FIG. 17A as a function of the irradiant light intensity. The experimental and analytical values open circuit voltage, V_{oc}, obtained by solving Equation (15) in Maple® are plotted in FIG. 17B as a function of the irradiant light intensity. As can be seen from FIGS. 17A and 17B, the results are in good agreement within ±10% variation.

Validation of Piezoelectric Microactuator Characteristic

The experimental results of the voltage-deflection behavior of the piezoelectric thin film microactuator were validated through a finite element (FE) model of the actuator. An axisymmetric FE model of the actuator was created in ANSYS® using PLANE183 structural elements for the non-piezoelectric materials and PLANE223 2D-coupled field elements with piezoelectric capability for the PZT film (a total of 1400 elements). The material properties of the constitutive material layers used in the FE analysis are given in Table. II. The geometry of the actuator diaphragm was modeled to match the profile of the actual fabricated prototype actuator, which comprised a fillet near the edges due to the DRIE etching process. The diaphragm thicknesses at various locations along a diameter of the diaphragm were measured using an optical microscope equipped with a digital camera and the corresponding geometry was created using a b-spline curve in the model. The center deflections of the actuator were computed via the FE model for input voltages ranging from 100 mV to 800 mV. Analytical and Experimental results were in excellent agreement. Ideally, if the diaphragm were perfectly flat, the actuator would yield larger deflections as predicted analytically (the 800 μm radius diaphragm with edge fillet effect is determined to be equivalent to a flat diaphragm with an effective radius of 721 μm).

Validation of the Integrated Device Characteristic

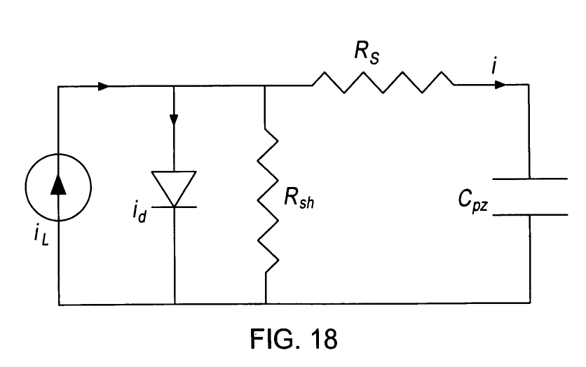

An analytical model of the integrated microactuator was developed to validate the experimentally determined input-output behavior (irradiance vs. deflection characteristic). In this model, the PZT actuator is idealized as a capacitor across the solar cell as illustrated in FIG. 18. This idealization is valid for sub-resonant operation of the piezoelectric material and adequate under the small voltage loads even though it neglects the electro-mechanical coupling in the piezoelectric material.

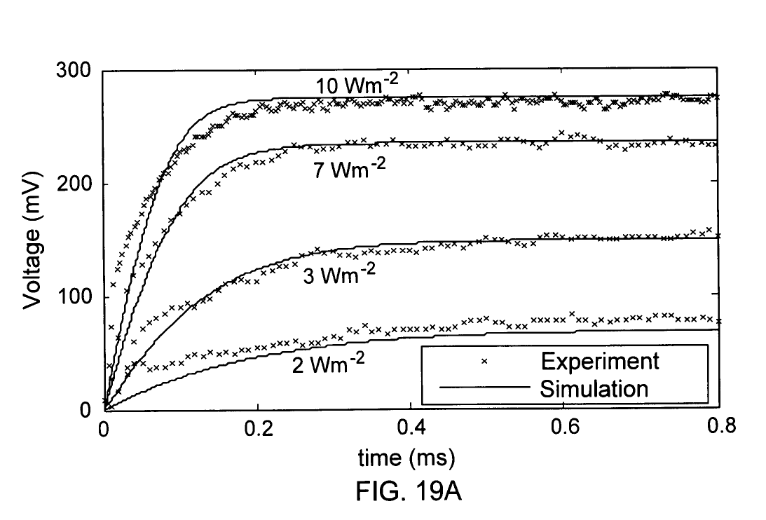

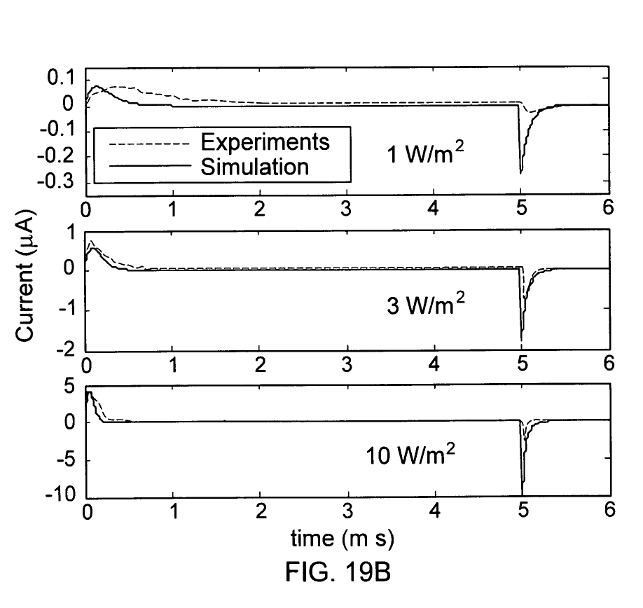

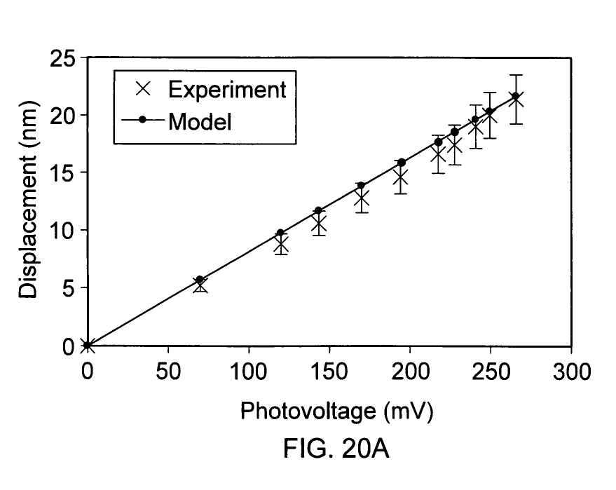

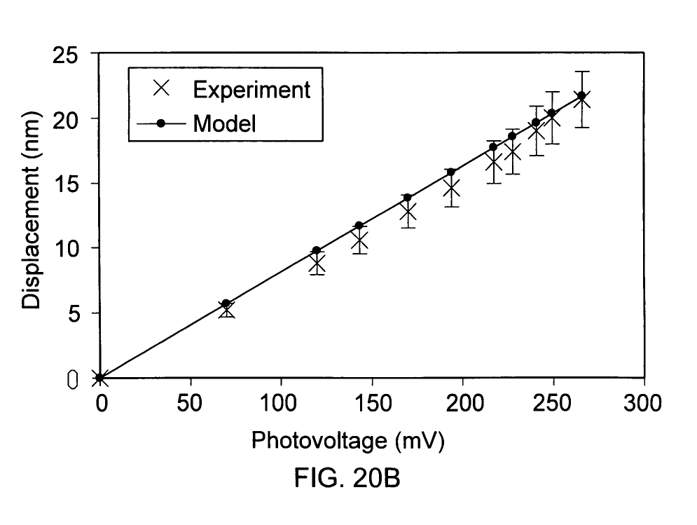

The governing differential equation for this system can be expressed using the averaged solar cell parameters given in (3) as,

$$\frac{d q ( t )}{d t} = {i}_{L} G - {I}_{0}^{*} ( {e}^{[ ( \frac{q ( t )}{{C}_{pz}} + \frac{d q ( t )}{d t} {R}_{s}^{*} ) + \frac{d q ( t )}{d t} {R}_{s}^{*} ] / {n}^{*} {V}_{T}} - 1 ) - \frac{1}{{R}_{sh}^{*}} [ ( \frac{q ( t )}{{C}_{pz}} + \frac{d q ( t )}{d t} {R}_{s}^{*} ) + \frac{d q ( t )}{d t} {R}_{s}^{*} ] \qquad ( 16 )$$

where, q(t) is the charge flow as a function of time, C_{pz} is the capacitance of the PZT thin film. The value of the capacitance C_{pz} was measured to be 11.78 nF. Equation (16) was numerically solved using SIMULINK®. The current, voltage and the electrical power were computed with a 100 Hz square irradiance signal of 1, 3, 7 and 10 W/m² magnitudes. FIG. 19A shows the voltage build-up across the actuator (capacitor) as irradiance is turned on and then drop when the irradiance is turned off. The modeled results are in good agreement with the experiments for the state when the full voltage is build-up (the simulated results show a slower rate of voltage build-up as the model does not capture the initial state of the solar cell, which is forward-biased during the experiment when the light signal is switched off). FIG. 19B shows the current flowing in the circuit in a positive direction to charge the PZT thin film capacitor when the light is on and reversing its direction when the light is off.

The experimental-model correlations of the input-output characteristics of the integrated actuator are shown in FIGS. 19A and 19B. In FIG. 19(A), the output displacements of the actuator are plotted as a function of the solar cell photovoltage, and in FIG. 19(B), the displacements are plotted directly as a function of the input irradiance on the solar cell. It can be seen that the experimental results and the results obtained from the models are in good agreement within a 10% margin of error. The plot in FIG. 19(B) shows that the actuator generates displacements of approximately 10 nm at an irradiance of 2 W/m² and over 20 nm for an irradiance of 10 W/m².

Response Time

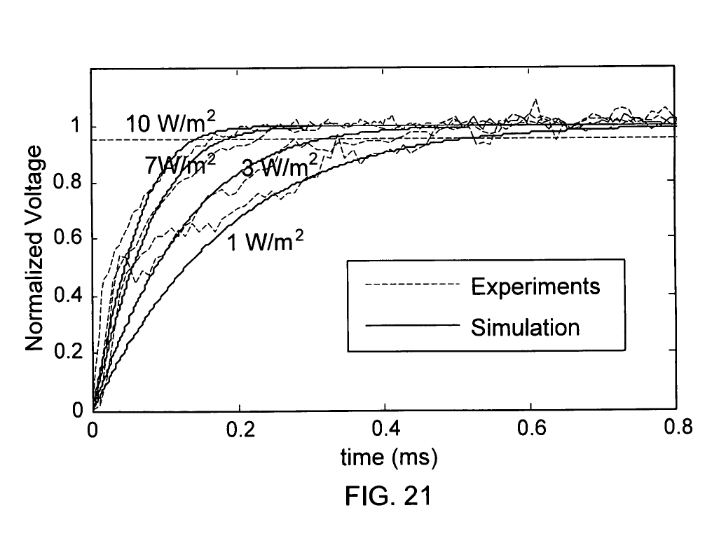

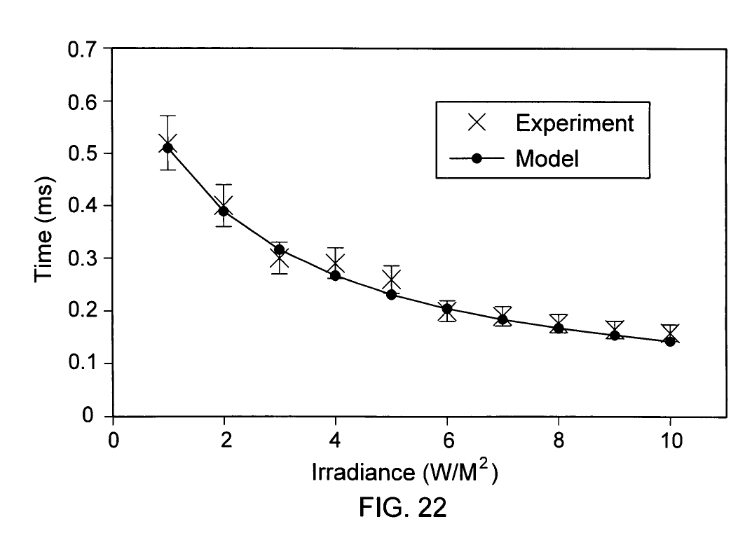

The microactuator response time is defined as the duration in which the microactuator deflection reaches 95% of its maximum deflection for a step input of light signal. Since direct measurement of this response time in terms of deflections was difficult due to instrumentation noise and significant response lag is due to the solar cell, the actuator response is characterized in terms of the time in which the photovoltage to reach 95% of its steady state voltage value. This response time, which is a good estimate of the actuator response for quasi-static deflections, is obtained by normalizing the photovoltage values at various irradiances determined in FIG. 19A with respect to their steady state voltages and plotted in FIG. 21. FIG. 22 shows the actuator's response time as a function of the input irradiance. The figures show that the response time decreases with increase in the irradiance as expected and there is a good agreement between the simulation model and the experiment.

The volume displacement and the block pressure for the microactuator are computed using an accurate analytical model for the deflections of the microactuator that can be solved in closed form. The volume displacement equation as a function of the applied voltage and pressure load on the microactuator can be expressed as a linear superposition of the displacements due to the voltage and pressure loads:

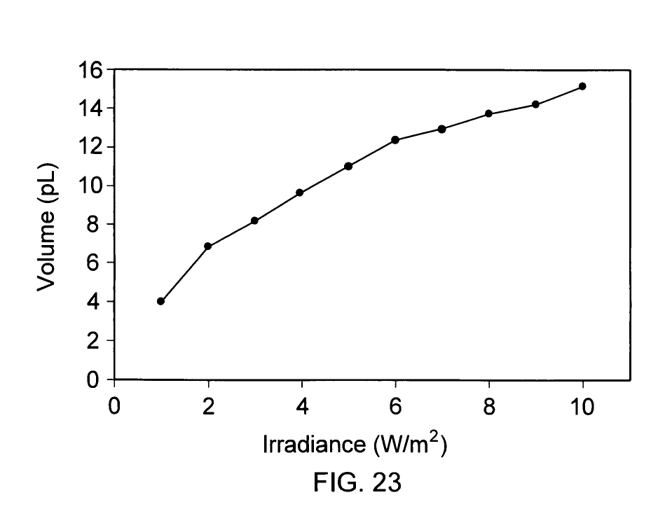

V_{disp}=CᵥV+C_{P}P (17)

where C_{V} is the voltage-induced volumetric compliance of the microactuator defined as the volume displacement per unit voltage load and C_{P} is the pressure-induced volumetric compliance defined as the volume displacement per unit pressure load, and V and P are applied voltage and pressure respectively. The volumetric compliances C_{V} and C_{P} are functions of the actuator geometry and the material properties. For the actuator material properties listed in Table II, the diaphragm effective radius 721 μm (corrected for the fillet edge effect), and the transverse piezoelectric coefficient of the PZT d₃₁=30 pm/V, the compliances of the actuator were C_{V}=0.569×10⁻¹³ m³/V and C_{P}=0.192×10⁻¹⁵ m³/Pa. The net volume displaced by the microactuator under pure light irradiance (i.e., P=0) calculated using Eq. (6), is plotted in FIG. 23 as a function of the input irradiance.

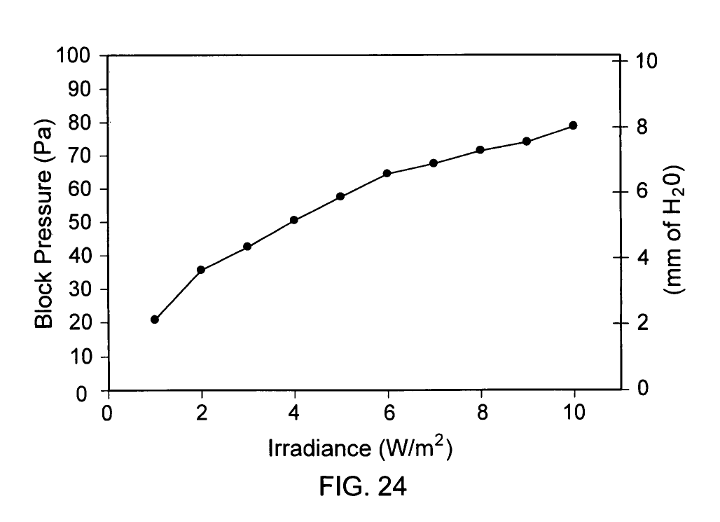

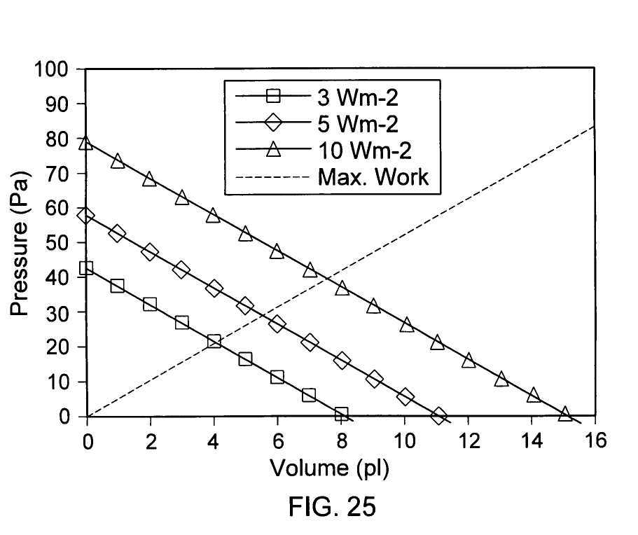

FIG. 24 shows the block-pressure of the actuator, P_{block}, calculated using Eq. (17) as a function of the irradiance. The actuator produces a maximum block-pressure of 78.8 Pa at 10 W/m² irradiance. The typical actuator performance curves are plotted in FIG. 25 for various irradiances of the input light.

In sum, the prototype optically powered microactuator was successfully fabricated, operated and characterized on a single chip via microfabrication. The actuator prototypes produced deflections of over 20 nm when irradiated by light irradiance of 10 W/m² with an actuation response time of less than 1 ms. The example prototype microactuator is capable of generating 15 pl in volume displacements and about 78.8 Pa in block-pressure at an irradiance of 10 W/m². Thus, the actuator could enable dispensing of chemicals from a dispenser in a retinal implant application that relies on chemical stimulation of the retinal neurons in response to the light stimulus naturally reaching the retina (<10 W/m²). If irradiance was not a limitation, the actuator could be redesigned to produce even greater deflections at higher irradiances that will benefit other applications seeking optically powered wireless actuation.

TABLE I
Solar Cell Model Parameters

| | Parameters at irradiance G | | | | | | | | | | |
|---|---|---|---|---|---|---|---|---|---|---|---|
| G (W/m²) | 1 | 2 | 3 | 4 | 5 | 6 | 7 | 8 | 9 | 10 | Averages |
| I_{L} (μA) | 4.66 | 8.94 | 11.01 | 14.81 | 19.27 | 24.72 | 27.50 | 31.43 | 34.58 | 41.16 | |
| I_{L}/G (μA(W/m²)⁻¹) | 4.66 | 4.47 | 3.67 | 3.70 | 3.85 | 4.12 | 3.93 | 3.93 | 3.84 | 4.12 | i_{L} = 4.03 μA |
| | | | | | | | | | | | (W/m²)⁻¹ |
| I₀ (μA) | 4.46 | 4.78 | 4.63 | 4.77 | 5.07 | 5.25 | 5.32 | 5.49 | 5.62 | 5.81 | I₀* = 5.12 μA |
| n | 5.00 | 5.00 | 5.00 | 5.00 | 5.00 | 5.00 | 5.00 | 5.00 | 5.00 | 5.00 | n* = 5.00 |
| Rₛₕ (KΩ) | 89.62 | 135.87 | 96.71 | 99.14 | 141.59 | 158.94 | 161.37 | 234.90 | 278.47 | 306.65 | Rₛₕ* = 170.33 KΩ |
| Rₛ (μΩ) | 67.64 | 0.04 | 43.09 | 15.16 | 14.41 | 0.52 | 49.04 | 45.54 | 6.89 | 33.82 | Rₛ* = 27.6 μΩ |
| I_{sc} (μA) | 4.66 | 8.94 | 11.01 | 14.81 | 19.27 | 24.72 | 27.50 | 31.43 | 34.58 | 41.16 | |
| Iₘₚ (μA) | 2.48 | 4.98 | 6.19 | 8.52 | 11.40 | 14.95 | 16.78 | 19.47 | 21.61 | 26.08 | |
| Vₘ (mV) | 41.78 | 69.75 | 79.84 | 96.17 | 112.21 | 127.82 | 134.73 | 143.87 | 149.84 | 161.11 | |
| Pₘ (μW) | 0.10 | 0.35 | 0.49 | 0.82 | 1.28 | 1.91 | 2.26 | 2.80 | 3.24 | 4.20 | |
| V_{oc} (mV) | 78.78 | 126.43 | 143.45 | 169.60 | 194.06 | 217.62 | 227.91 | 241.10 | 249.72 | 266.02 | |
| FF % | 28.19 | 30.73 | 31.29 | 32.63 | 34.22 | 35.53 | 36.08 | 36.97 | 37.49 | 38.37 | |

TABLE II
Actuator Material Properties

| Property | Si | Oxide | Pt | Au | PZT |
|---|---|---|---|---|---|
| Young's modulus (GPa) | 168.9 | 70 | 170 | 41.9 | 101 |
| Poisson's ratio | 0.182 | 0.3 | 0.38 | 0.42 | 0.45 |
| d_{3L}(pm/V) | | | | | −30 [3] |

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A light powered microactuator device, comprising:
a substrate;
a flexible structure formed by a portion of said substrate;
a solar cell formed integrally within said substrate; and
a thin film electroactive microactuator coupled to said flexible structure including an electroactive thin film and electrodes for establishing a potential across said thin film electroactive microactuator to induce a lateral strain therein and cause actuation of said flexible structure.

2. The device of claim 1, wherein said substrate comprises a silicon wafer and said solar cell comprises a p-n junction formed by doped regions in said silicon wafer.

3. The device of claim 2, wherein said flexible structure comprises a flexible membrane and said doped regions surround said film membrane.

4. The device of claim 2, wherein said electrodes comprise:
a top electrode formed over said electroactive thin film and said doped regions, wherein said top electrode has a grid pattern over said doped regions; and
a bottom electrode disposed on an opposite side of said electroactive thin film and isolated from said top electrode.

5. The device of claim 1, wherein said flexible structure comprises a flexible membrane that is disposed over an enclosed volume.

6. The device of claim 5, wherein fluid is contained in said enclosed volume and an outlet is disposed to eject fluid in response to actuation of said flexible membrane.

7. A retinal prosthesis device, the device comprising a light powered microactuator device according to claim 5, and a neurotransmitter chemical fluid contained in the enclosed volume.

8. A light powered microactuator device, comprising:
a substrate;
a flexible structure formed by a portion of said substrate; and
means for actuating said flexible structure in response to illumination, said means for actuating being formed integrally within said substrate.

9. The device of claim 8, further comprising means for containing fluid and means for ejecting fluid in response to actuation of said flexible structure.

10. A light powered microactuator device, comprising:
a substrate;
a flexible structure made of a passive material formed in a portion of said substrate;
a photovoltaic device formed integrally within said substrate; and
an electroactive material layer coupled to said flexible structure with electrodes patterned on either side of the active material layer for applying a electrical potential received from said photovoltaic device across said electroactive material layer to thereby induce a mechanical strain in said flexible structure and cause mechanical actuation thereof.
